(19) United States
(12) Patent Application Publication
(10) Pub. No.: US 2019/0010236 A1
(43) Pub. Date: Jan. 10, 2019

(54) METHODS AND COMPOSITIONS FOR DECTIN-2 STIMULATION AND CANCER IMMUNOTHERAPY

(71) Applicant: The Board of Trustees of the Leland Stanford Junior University, Standford, CA (US)

(72) Inventors: Justin Kenkel, Mountain View, CA (US); Carolyn R. Bertozzi, Stanford, CA (US); Edgar George Engleman, Atherton, CA (US)

(21) Appl. No.: 16/066,154

(22) PCT Filed: Dec. 28, 2016
(86) PCT No.: PCT/US2016/068967

§ 371 Date: Jun. 26, 2018

Related U.S. Application Data
(60) Provisional application No. 62/272,475, filed on Dec. 29, 2015.

(51) Int. Cl. C07K 16/28; A61P 35/00; C07K 14/705; C07K 14/47

(52) U.S. Cl. CPC C07K 16/2851; C07K 16/2878; A61K 31/437; C07K 14/7056; C07K 14/4727; A61P 35/00

(57) ABSTRACT

Provided are methods and compositions for treating an individual with cancer by administering to the individual a composition that includes a Dectin-2 stimulating agent that stimulates Dectin-2 signaling in myeloid cells (e.g., induces Dectin-2 clustering on the cell surface), thereby stimulating an anti-cancer immune response in the individual. In some cases, the myeloid cells are tumor-associated myeloid (TAM) cells. Methods and compositions are also provided for: treating an individual with cancer via contacting a cancer cell from the individual with an alpha-mannosidase class 1 inhibitor (e.g., to increase the display and/or density of terminal mannose/mannobiose residues on the surface of target cells) in vitro or ex vivo and introducing the contacted cancer cell into the individual; stimulating an antigen presenting cell (APC) via contacting a cancer cell with an alpha-mannosidase class 1 inhibitor and contacting the APC with the inhibitor-contacted cancer cell; and stimulating an APC via contacting it with a subject Dectin-2 stimulating agent.

### CROSS REFERENCE

This application claims benefit of U.S. Provisional Patent Application No. 62/272,475, filed Dec. 29, 2015, which application is incorporated herein by reference in its entirety.

### INTRODUCTION

Despite the ability of the immune system to detect subtle differences between tumor cells and normal tissues, cancers tend to grow and spread, often leading to the death of their hosts. An adaptive immune response to tumor associated antigens (TAA) can occur in this setting, resulting in tumor control or regression. However, aggressive tumors eventually escape from immune control via immunoediting and other mechanisms that suppress antitumor immune cells and mediators.

Immune cells are a major component of the stromal compartment in most cancers, and play a critical role in shaping tumor development and progression. Cancer immunotherapies (e.g. checkpoint inhibitors, cancer vaccines, CAR T cells, etc.) have proven effective in several cancers; however, many patients and types of cancer fail to respond to these kinds of interventions, suggesting that alternative immunotherapeutic strategies should be explored.

Myeloid cells, which include granulocytes, monocytes (Mo), macrophages (Mϕ), and dendritic cells (DC), are particularly abundant in most tumors and have been shown to promote disease progression in various ways and across a range of malignancies. Despite this, relatively few therapies in clinical development are directed toward this major stromal cell population, and most of them aim to inhibit the accumulation of these cells rather than modulate their activity.

There is need in the art for compositions and methods for reprogramming tumor-associated myeloid cells in order to achieve therapeutic benefits.

### SUMMARY

Provided are methods and compositions for treating an individual with cancer by administering to the individual a composition that stimulates Dectin-2 signaling in myeloid cells (e.g., by inducing Dectin-2 clustering on the myeloid cell surface), thereby stimulating an anti-cancer immune response in the individual. In some cases, the myeloid cells are tumor-associated myeloid (TAM) cells.

Agents that stimulate Dectin-2 signaling in myeloid cells are referred to herein as “Dectin-2 stimulating agents,” which can directly stimulate Dectin-2 signaling in myeloid cells (e.g., by binding to Dectin-2 on the surface of myeloid cells) or can indirectly stimulate Dectin-2 signaling (e.g., by increasing the display and/or density of terminal mannose/mannobiose/oligomannose residues on the surface of target cells such as cancer cells). Examples of Dectin-2 stimulating agents include but are not limited to: (a) non-plant derived naturally existing ligands for Dectin-2 (e.g., fungal cell wall extracts such as those from Malassezia furfur (M. furfur) and/or Candida albicans; a naturally occurring oligomannose glycopolypeptide such as a mannobiose-rich glycoprotein, e.g., an O-linked and/or N-linked mannobiose-rich glycoprotein; mannan polysaccharide; and other oligomannose glycans such as can be found in mannan extract from S. cerevisiae; and the like); (b) synthetic glycopolymer ligands for Dectin-2 (or mimetics thereof) (e.g., glycopolypeptides such as oligomannose glycopolypeptides) (e.g., not conjugated to a targeting agent, or conjugated to a targeting agent such as a cancer cell targeting agent, e.g., an antibody that specifically binds to a cancer cell antigen); (c) Dectin-2 stimulating anti-Dectin-2 antibodies (e.g., an anti-Dectin-2 antibody such as a soluble antibody or one that is immobilized on a solid support, a multivalent anti-Dectin-2 antibody that also binds specifically to a cancer antigen, and the like); and (d) alpha-mannosidase class 1 inhibitors (e.g., kifunensine) which can increase the display and/or density of terminal mannose/mannobiose residues on the surface of target cells (e.g., cancer cells) and thereby increase the sensitivity/strength of an immune response to the cancer being treated.

Provided are methods and compositions for treating an individual with cancer where the methods include contacting a cancer cell (e.g., from the individual) with an alpha-mannosidase class 1 inhibitor in vitro or ex vivo (e.g., to increase the display and/or density of terminal mannose/mannobiose residues on the surface of the cancer cell), and introducing the contacted cancer cell into the individual. In some cases, the cancer cell is autologous to the individual (i.e., the cancer cell was isolated from the individual or was derived from a cell isolated from the individual). Also provided are methods and compositions for stimulating an antigen presenting cell (APC) (e.g., a dendritic cell, a macrophage, a B-cell) where the methods include contacting a cancer cell with an alpha-mannosidase class 1 inhibitor in vitro or ex vivo (e.g., to increase the display and/or density of terminal mannose/mannobiose residues on the surface of the cancer cell) and contacting the APC with the inhibitor-contacted cancer cell. Also provided are methods and compositions for stimulating an antigen presenting cell (APC) (e.g., a dendritic cell, a macrophage, a B cell) where the methods include contacting the APC with a composition comprising a subject direct Dectin-2 stimulating agent (e.g., a direct Dectin-2 stimulating agent).

Provided is a method of treating an individual with cancer where the method includes: administering to the individual a Dectin-2 stimulating composition comprising one or more Dectin-2 stimulating agents selected from: (a) a non-plant derived naturally existing ligand for Dectin-2; (b) a synthetic Dectin-2 stimulating glycopolymer or mimetic thereof (e.g., a glycopolypeptide); (c) a Dectin-2 stimulating anti-Dectin-2 antibody, and (d) an alpha-mannosidase class 1 inhibitor, where Dectin-2 signaling is stimulated in myeloid cells thereby stimulating an anti-cancer immune response in the individual.

In some cases, the Dectin-2 stimulating composition includes mannan polysaccharide (see, e.g., mannan extract from S. cerevisiae). In some cases, the Dectin-2 stimulating composition includes a fungal cell wall extract comprising one or more naturally existing glycoproteins that stimulate Dectin-2 signaling. In some cases, the Dectin-2 stimulating composition includes a non-plant derived naturally existing Dectin-2 ligand that is a glycoprotein isolated from a fungal cell wall extract. In some cases, the cell wall extract is an M. furfur cell wall extract. In some cases, the Dectin-2 stimulating composition includes an extract from one or more of: Candida albicans, Schistosoma mansoni, Mycobacterium tuberculosis, and Dermatophagoides farina, wherein the extract comprises one or more naturally existing glycoproteins that stimulate Dectin-2 signaling. In some cases, the Dectin-2 stimulating composition includes an anti-Dectin-2 antibody (e.g., a soluble antibody such as a soluble monoclonal antibody). In some cases, the Dectin-2 stimulating composition includes an anti-Dectin-2 antibody immobilized on a solid support. In some cases, the solid support is a nanoparticle or microparticle. In some cases, the Dectin-2 stimulating composition includes kifunensine, which is an alpha-mannosidase class 1 inhibitor.

In some cases, said administration includes local administration. In some cases, said administration includes systemic administration. In some cases, said administration includes co-administration with one or more of: a CD40 agonist, GM-CSF, TNFα, and IFNγ.

Also provided is a method of treating an individual with cancer where the method includes: contacting a cancer cell from the individual with an alpha-mannosidase class 1 inhibitor in vitro or ex vivo, and introducing the contacted cancer cell into the individual. In some cases, the contacted cancer cell is administered locally into a tumor of the individual.

Also provided is a method of stimulating an antigen presenting cell (APC) where the method includes: (a) contacting in vitro or ex vivo a cancer cell with an alpha-mannosidase class 1 inhibitor to produce an inhibitor-contacted cancer cell; and (b) contacting an APC with the inhibitor-contacted cancer cell. In some cases, the alpha-mannosidase class 1 inhibitor is an RNAi agent or gene editing agent that specifically reduces expression of one or more proteins selected from: MAN1B1, MAN1A1, MAN1A2, and MAN1C1. In some cases, the alpha-mannosidase class 1 inhibitor is selected from: kifunensine and 1-deoxymannojirimycin. In some cases, the APC is a dendritic cell or a macrophage. In some cases, the method includes, after step (b), introducing the contacted APC into the individual. In some cases, the method includes, after step (b), contacting a T cell with the contacted APC, thereby stimulating the T cell. In some cases, the method includes introducing the stimulated T cell into the individual. In some cases, the T cell is autologous to the individual

Also provided is a method of stimulating an antigen presenting cell (APC) where the method includes: contacting an APC with a Dectin-2 stimulating composition comprising one or more Dectin-2 stimulating agents selected from: (i) a non-plant derived naturally existing ligand for Dectin-2; (ii) a synthetic Dectin-2 stimulating glycopolymer or mimetic thereof (e.g., a glycopolypeptide); and (iii) a Dectin-2 stimulating anti-Dectin-2 antibody, at a dose and for a period of time sufficient to enhance Dectin-2 signaling in the APC, thereby generating a stimulated APC. In some cases, the method includes contacting the stimulated APC with a cancer antigen to produce an antigen-contacted APC. In some cases, the cancer antigen is present in a cancer cell lysate or is part of a cancer cell. In some cases, the method includes introducing the stimulated APC or the antigen-contacted APC into an individual. In some cases, the cancer antigen is from an individual with cancer and the method comprises introducing the antigen-contacted APC into the individual. In some cases, the APC is autologous to the individual. In some cases, the method includes contacting a T cell with the antigen-contacted APC. In some cases, the method includes introducing the contacted T cell into an individual. In some cases, the T cell is autologous to the individual. In some cases, the antigen-contacted APC is autologous to the individual. In some cases, the individual has cancer.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1B. Tumor-associated myeloid cells express high levels of Dectin-2. (FIG. 1A-1B) Tissues from murine and human (Hu) pancreatic ductal adenocarcinoma (PDAC) were stained with the indicated antibodies and imaged by fluorescence (FIG. 1A) or light (FIG. 1B) microscopy. (FIG. 1A) Primary pancreatic tumor tissues and metastatic liver samples from immunocompetent mice injected with the LMP or Panc02 murine tumor cell line. LMP cells (originally obtained from Pdx1-Cre; Kras^{LSL-G12D/+}; Trp53^{LSL-R172H/+} mice and used throughout these studies) are marked by the accumulation of mutant p53. (FIG. 1B) Primary tumor and metastatic tissues from human PDAC and a genetically engineered mouse model (GEMM) of PDAC (Pdx1-Cre; Kras^{LSLG12D/+}; Cdkn2a^{-/-}. Scale bar, 100 μm.

FIG. 2A-2G. A natural Dectin-2 agonist activates tumor-associated myeloid cells and induces antitumor immune responses. (FIG. 2A-2C) Murine bone marrow monocytes were cultured with PDAC-conditioned medium to generate TAM-like cells. Cytokine production and costimulatory molecule expression were analyzed following overnight stimulation with a cell wall extract from M. furfur (furfurman). In some experiments (FIG. 2B), cells were pretreated with Dectin-2 blocking antibody. ND, not detected. (FIG. 2D) Subcutaneous PDAC tumors were injected with furfurman or vehicle on two consecutive days and then analyzed by flow cytometry on day 3. Total CD3⁺ T cells among tumor-infiltrating immune cells are gated. (FIG. 2E-2G) Mice bearing subcutaneous PDAC tumors were injected intratumorally with furfurman (+/−IFNγ) or vehicle on days 7 and 10 post-tumor implantation, and treated systemically with checkpoint inhibitors (FIG. 2E), gemcitabine (Gem) (FIG. 2F), or CD40 agonistic antibody (i.p., q3d starting on day 7). Mean tumor volumes±SEM are displayed (n=3-5 mice/group). *, p<0.05; **, p<0.01; ***, p<0.001; ****, p<0.0001 by one-way ANOVA with post hoc Tukey's test, with results shown for tumor volumes at the last time point.

FIG. 3A-3F. Natural Dectin-2 ligands activate mouse and human cells and have anticancer effects in multiple tumor types. (FIG. 3A) TNFα production by PDAC TAM pretreated with the indicated antibodies and then stimulated overnight with plate-bound S. cerevisiae mannan. (FIG. 3B, FIG. 3C) TNFα production by human monocytes pretreated with GM-CSF and then stimulated with furfurman (FIG. 3B) or mannan (FIG. 3C). Mean±SEM for n=3 donors shown. (FIG. 3D-3F) Mice bearing s.c. PDAC (FIG. 3D), lung adenocarcinoma (FIG. 3E), or CT26 colon carcinoma were treated with mannan (i.v.) and/or a combination of αCTLA-4 and αPD-1 antibodies (i.p.) starting 6-9 days after tumor implantation. Mean tumor volumes±SEM are shown (n=3-5 per group). *, p<0.05; **, p<0.01; ***, p<0.001; ****, p<0.0001 by unpaired Student's t-test (FIG. 3B) or two-way ANOVA with post hoc Tukey's test (FIG. 3D-3F).

FIG. 4A-4D. GM-CSF induces Dectin-2 expression and sensitizes tumors to Dectin-2 stimuli. (FIG. 4A-4C) Murine (FIG. 4A, FIG. 4B) and human (FIG. 4C) monocytes were cultured for 24 hr in media supplemented or not with GM-CSF (50 ng/mL) prior to flow cytometric analysis of Dectin-2 expression (FIG. 4A, FIG. 4C) or stimulation with furfurman and analysis of TNFα production (FIG. 4B). (FIG. 4C) Mean MFI±SEM for n=3 donors displayed. (FIG. 4D) Mice bearing s.c. CT26 tumors were treated with mannan (i.v.) and/or GM-CSF (i.t.) starting on day 6 post-tumor implantation. Mean tumor volumes±SEM are displayed (n=3-4 per group). *, p<0.05; **, p<0.01 by Student's t-test (FIG. 4C) or two-way ANOVA with post hoc Tukey's test (FIG. 4D).

FIG. 5A-5D. Mannosidase inhibition with kifunensine induces high-mannose glycan display and increases tumor cell immunogenicity. (FIG. 5A-5C) PDAC cells were treated with kifunensine for 3 days prior to flow cytometric analysis (FIG. 5A) or coculture with TAM (FIG. 5B, FIG. 5C). (FIG. 5A) PDAC cells stained with the mannose-binding lectin, concavalin A. (FIG. 5B, FIG. 5C) Pretreated PDAC cells were labeled with CSFE and cocultured overnight with TAM to assess cytokine production (FIG. 5B) and tumor cell uptake (FIG. 5C) by Dectin-2-expressing TAM. In some experiments, TAM were treated with Dectin-2-blocking antibodies prior to coculture with PDAC cells (FIG. 5C). *, p<0.05 by Student's t-test. (FIG. 5D) Subcutaneous PDAC tumors from kifunensine-treated mice (i.p., q2d for 7 days) were analyzed by flow cytometry. CD8 T cells (CD8⁺CD90⁺) among total tumor-infiltrating immune cells are gated.

FIG. 6A-6B. Dectin-2 antibodies (e.g., soluble or immobilized Dectin-2 antibodies) activate tumor-associated myeloid cells. (FIG. 6A, FIG. 6B) Murine bone marrow monocytes were cultured with PDAC-conditioned medium to generate TAM-like cells and then seeded in wells coated with antibodies directed against various cell surface molecules. Cytokine production (FIG. 6A) and costimulatory molecule expression (FIG. 6B) were assessed after 18 hr.

FIG. 7A-7C. Design of synthetic glycopolymers capable of activating Dectin-2. (FIG. 7A) Polyfunctional glycopolymers can be designed to activate Dectin-2 within the tumor environment. Glycopolymers display Dectin-2-binding glycans on a polymer backbone with optimal spacing for receptor clustering and activation. Other functionalities can be integrated along the polymer backbone or as end-groups for targeting, imaging, stimulating additional cellular pathways, or pharmacological optimization. (FIG. 7B) An example of one embodiment in which mannobiosyl groups serve as Dectin-2 ligands attached to serine residues within a polypeptide backbone. These glycopolypeptides can be synthesized by polymerization of amino acid N-carboxyanhydrides (NCAs). (FIG. 7C) A schematic example of one possible conjugation strategy (which was used successfully) to attach synthetic glycopeptides to an antibody, generating a glycopeptide-antibody conjugate. Lysine residues on the antibody were treated with NHS-cyclooctyne compounds, followed by bioorthogonal covalent reaction with azide terminal glycopeptides.

FIG. 8A-8C. Synthetic mannobiose glycopolymers and glycoconjugates activate myeloid cells for therapeutic effect. (FIG. 8A, FIG. 8B) TNFα production by PDAC TAM pretreated with the indicated antibodies and then stimulated with (FIG. 8A) mannose (Man1) or mannobiose (Man2) glycopolymers of different glycan densities (35% or 65%) or (FIG. 8B) αEpCAM antibodies coupled to lactose (Lac) or Man2 glycopolymers. (FIG. 8C) Mice bearing s.c. PDAC tumors were treated or not with Man2 glycopolymers (i.v.) starting 10 d following tumor implantation. Mean tumor volumes±SEM are shown (n=3-5 per group). ***, p<0.001; ****, p<0.0001 by two-way ANOVA with post hoc Tukey's test.

### DETAILED DESCRIPTION OF THE EMBODIMENTS

### I. Introduction

Described herein are methods, compositions, and kits for the treatment of cancer. Some of the methods, compositions, and kits are based on a discovery by the inventors that Dectin-2 stimulation is surprisingly effective at treating cancer. For example, the inventors have discovered that stimulation of Dectin-2 signaling in myeloid cells (tumor-associated myeloid cells such as dendritic cells and macrophages) can enhance/stimulate an immune response to cancer. The inventors envision at least two general categories of Dectin-2 stimulating agents that can be used to stimulate Dectin-2 signaling in myeloid cells (e.g., tumor-associated myeloid cells) in order to enhance/stimulate an immune response to cancer: (1) direct Dectin-2 stimulating agents, which are systemically and/or locally administered agents that bind to Dectin-2 (e.g., specifically bind to Dectin-2) on myeloid cells and stimulate Dectin-2 signaling; and (2) indirect Dectin-2 stimulating agents, which are systemically and/or locally administered agents (e.g., alpha-mannosidase class 1 inhibitors such as kifunensine, gene editing agents and/or RNAi agents that can reduce mannosidase expression and/or activity, etc.) that increase the display and/or density of terminal mannose/mannobiose residues on the surface of target cells (e.g., cancer cells), and thus increase the amount of Dectin-2 ligands on the surface of the target cells.

In some cases, a direct Dectin-2 stimulating agent (i.e., an agent that binds to Dectin-2 and stimulates Dectin-2 signaling in myeloid cells, e.g., an antigen binding region of an anti-Dectin-2 antibody, a glycopolymer such as a glycopolypeptide that binds to Dectin-2, etc.) is conjugated to a targeting agent that targets the Dectin-2 stimulating agent to a target cell (e.g., a cancer cell) such that the Dectin-2 stimulating agent is displayed on the surface of the target cell. Because such an agent can bind to two different target molecules (e.g., a target molecule such as a cancer antigen on the surface of a cancer cell, and Dectin-2 on the surface of a myeloid cell), a Dectin-2 stimulating agent that is conjugated to a targeting agent is sometimes referred to herein as a multivalent Dectin-2 stimulating agent. Compositions and kits for practicing the methods of the disclosure are also provided.

Before the present methods and compositions are described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed

### II. Definitions

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides). For example, an anti-Dectin-2 antibody preferentially binds to Dectin-2 relative to other available antigens. In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁵ M or less, 10⁻⁹ M or less, 10⁻¹⁵ M or less, 10¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding. For example increased binding affinity can be indicated by a lower K₀. In some cases, increased binding affinity is correlated with a lower K₀.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member).

The term “specific binding agent” as used herein refers to any agent that specifically binds a biomolecule (e.g., a marker such as a nucleic acid marker molecule, a protein marker molecule, etc.). In some cases, a “specific binding agent” for a marker molecule (e.g., a dendritic cell marker molecule) is used. Specific binding agents can be any type of molecule. In some cases, a specific binding agent is an antibody or a fragment thereof. In some cases, a specific binding agent is a nucleic acid probe (e.g., an RNA probe; a DNA probe; an RNA/DNA probe; a modified nucleic acid probe, e.g., a locked nucleic acid (LNA) probe, a morpholino probe, etc.; and the like).

As used herein, a “marker molecule” does not have to be definitive (i.e., the marker does not have to definitely mark the cell as being of a particular type). For example, the expression of a marker molecule by a cell can be indicative (i.e., suggestive) that the cell is of a particular cell type. For example, if 3 cell types (type A, type B, and type C) express a particular marker molecule (e.g., a particular mRNA, a particular protein, etc.), expression of that marker molecule by a cell cannot necessarily be used by itself to definitively determine that the cell is a type A cell. However, expression of such a marker can suggest that the cell is a type A cell. In some cases, expression of such a marker, combined with other evidence, can definitively show that the cell is a type A cell. As another illustrative example, if a particular cell type is known to express two or more particular marker molecules (e.g., mRNAs, proteins, a combination thereof, etc.) then the expression by a cell of one of the two or more particular marker molecules can be suggestive, but not definitive, that the cell is of the particular type in question. In such a case, the marker is still considered a marker molecule.

“Antibody” refers to a polypeptide comprising an antigen binding region (including the complementarity determining region (CDRs)) from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding site. There are four IgG subclasses (IgG1, 2, 3, and 4) in humans, named in order of their abundance in serum (IgG1 being the most abundant). Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

The term “antibody” is used in the broadest sense and encompasses monoclonal antibodies (including full length monoclonal antibodies), polyclonal antibodies, multispecific antibodies (e.g., bispecific antibodies), and antibody fragments so long as they exhibit the desired biological activity (e.g., specifically binds to a target antigen). “Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety; (4) nanobodies comprising single Ig domains from non-human species or other specific single-domain binding modules; and (5) multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). In some cases, an antibody (e.g., an anti-Dectin-2 antibody) is a humanized antibody (e.g., can be an IgG4 isotype humanized antibody, e.g., an IgG4 isotype antibody having a mutation in the hinge region such as the S241P mutation that reduces heterogeneity sometimes found in chimeric mouse/human IgG4 antibodies)(e.g., see Angal et al., Mol Immunol. 1993 January; 30(1):105-8).

As used in this disclosure, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “polymer” refers to an oligomer made of monomer building blocks and can constitute a linear, branched or dendrimeric structure. For example, a “glycopolymer” is a polymeric structure that includes sugar building blocks, and a “glycopolypeptide” is a polymeric structure that includes sugar and amino acid building blocks.

As used herein, the term “APC” or “antigen presenting cell” refers to a cell that expresses major histocompatibility complex class II (MHC class II) proteins on its cell membrane surface and is capable of presenting antigens in complex with MHC class II to T-cells, thereby activating T-cells to the presented antigens. In some embodiments, the APC is a dendritic cell. In some embodiments, the APC is a macrophage. In some embodiments, the APC is a B-cell. In some embodiments, the APC is a dendritic cell, macrophage, or B-cell. In some embodiments, the APC is a dendritic cell or a macrophage. In some embodiments, the APC is a dendritic cell or a B-cell. In some cases, the APC is not a macrophage. In some cases, the APC is not a B-cell.

As used herein, the term “myeloid cell” encompasses granulocytes, monocytes (Mo), macrophages (Mϕ), and dendritic cells (DC). These cells are often abundant in tumors. Thus, myeloid cells are sometimes referred to herein as a tumor-associated myeloid (TAM) cells.

The terms “passaging” or “passage” (i.e., splitting or split) in the context of cell culture are known in the art and refer to the transferring of a small number of cells into a new vessel. Cells can be cultured if they are split regularly because it avoids the senescence associated with high cell density. For adherent cells, cells are detached from the growth surface as part of the passaging protocol. Detachment is commonly performed with the enzyme trypsin and/or other commercially available reagents (e.g., TrypLE, EDTA (Ethylenediaminetetraacetic acid), a policemen (e.g., a rubber policemen) for physically scrapping the cells from the surface, etc.). A small number of detached cells (e.g., as few as one cell) can then be used to seed a new cell population, e.g., after dilution with additional media. Therefore, to passage a cell population means to dissociate at least a portion of the cells of the cell population, dilute the dissociated cells, and to plate the diluted dissociated cells (i.e., to seed a new cell population).

The terms “media” and “medium” are herein used interchangeably. Cell culture media is the liquid mixture that baths cells during in vitro culture.

The term “population”, e.g., “cell population” or “population of cells”, as used herein means a grouping (i.e., a population) of two or more cells that are separated (i.e., isolated) from other cells and/or cell groupings. For example, a 6-well culture dish can contain 6 cell populations, each population residing in an individual well. The cells of a cell population can be, but need not be, clonal derivatives of one another. A cell population can be derived from one individual cell. For example, if individual cells are each placed in a single well of a 6-well culture dish and each cell divides one time, then the dish will contain 6 cell populations. A cell population can be any desired size and contain any number of cells greater than one cell. For example, a cell population can be 2 or more, 10 or more, 100 or more, 1,000 or more, 5,000 or more, 10⁴ or more, 10⁵ or more, 10⁶ or more, 10⁷ or more, 10⁸ or more, 10⁹ or more, 10¹⁰ or more, 10¹¹ or more, 10¹² or more, 10¹³ or more, 10¹⁴ or more, 10¹⁵ or more, 10¹⁶ or more, 10¹⁷ or more, 10¹⁸ or more, 10¹⁹ or more, or 10²⁰ or more cells.

The term “plurality” as used herein means greater than one. For example, a plurality can be 2 or more, 5 or more, 10 or more, 25 or more, 50 or more, 100 or more, 500 or more, 1,000 or more, 2,000 or more, 5,000 or more, 10⁴ or more, 10⁵ or more, 10⁶ or more, 10⁷ or more, etc.

“Dectin-2” is a type II membrane receptor with an extracellular C-type lectin-like domain fold. Unlike Dectin-1, Dectin-2 lacks an immunoreceptor tyrosine-based activation motif (ITAM) in its cytoplasmic domain. Human Dectin-2 (NCBI reference sequence NP_001007034.1) is also known as CLEC6A, “C-type lectin domain family 6 member A”, CLEC4N, and CLECSF10. The protein sequence of human Dectin-2 is:

(SEQ ID NO: 1)
MMQEQQPQSTEKRGWLSLRLWSVAGISIALLSACFIVSCVVTYHFTYGE
TGKRLSELHSYHSSLTCFSEGTKVPAWGCCPASWKSFGSSCYFISSEEK
VWSKSEQNCVEMGAHLVVFNTEAEQNFIVQQLNESFSYFLGLSDPQGNN
NWQWIDKTPYEKNVRFWHLGEPNHSAEQCASIVFWKPTGWGWNDVICET
RRNSICEMNKIYL

### III. Methods and Compositions

Aspects of the disclosure include methods and compositions for treating an individual with cancer (e.g., by administering to the individual a composition that stimulates Dectin-2 signaling in myeloid cells, e.g., by inducing Dectin-2 clustering on the cell surface, thereby stimulating an anti-cancer immune response in the individual). In some cases, the myeloid cells are tumor-associated myeloid (TAM) cells. Dectin-2 stimulation may be achieved by Dectin-2 clustering similar to that which occurs on the surface of a phagocyte upon contact with microbes displaying oligomannose glycans. Such stimulation can also be achieved with Dectin-2 antibodies (e.g., large, multivalent complexes of Dectin-2 antibodies) or ligands (e.g. mannobiose-rich glycopeptides, mannan polysaccharides, and/or other oligomannose glycans such as Man-9) that resemble microbes with a high density of Dectin-2 ligands, like M. furfur, S. cerevisiae, and other microbial species (e.g., several pathogenic species).

Agents that stimulate Dectin-2 signaling in myeloid cells (e.g., by increasing Dectin-2 density on the cell surface, e.g., by inducing Dectin-2 clustering on the cell surface) are referred to herein as “Dectin-2 stimulating agents.” Dectin-2 stimulating agents can be “direct” agents (e.g., they directly bind, e.g., specifically bind, to Dectin-2 on myeloid cells) or can be “indirect” agents (e.g., they increase the amount of Dectin-2 ligands on the surface of cells such as cancer cells). Examples of direct Dectin-2 stimulating agents include but are not limited to: (a) a non-plant derived naturally existing ligand for Dectin-2 (e.g., a mannan polysaccharide, a mannan extract, an oligomannose/high-mannose glycan, a fungal extract such as a cell wall extract from M. furfur, S. cerevisiae, C. albicans, and the like); (b) a synthetic Dectin-2 stimulating glycopolymer or mimetic thereof (e.g., a glycopolypeptide) (e.g., that directly binds to Dectin-2 on myeloid cells, or that is conjugated to an antibody that specifically binds to a cancer antigen); and (c) a Dectin-2 stimulating anti-Dectin-2 antibody (e.g., an anti-Dectin-2 antibody—in some cases soluble and in some cases immobilized on a solid support, a multivalent anti-Dectin-2 antibody, e.g., one that also binds specifically to a cancer antigen, etc.).

In some cases, a direct Dectin-2 stimulating agent can be used to contact a myeloid cell (e.g., in vitro, ex vivo, or in vivo, e.g., by administering the agent to an individual), thereby triggering (stimulating) Dectin-2 signaling in the myeloid cell. In some cases, a direct Dectin-2 stimulating agent can be conjugated to a cancer cell binding agent (e.g., an antibody against a tumor antigen), thus increasing the level of the direct Dectin-2 stimulating agent on the surface of the target cell (e.g., the cancer cell).

Examples of indirect Dectin-2 stimulating agent include but are not limited to: alpha-mannosidase class 1 inhibitors (e.g., kifunensine); and gene editing agents and/or RNAi agents that can reduce mannosidase expression and/or activity; all of which can increase the display and/or density of terminal mannose/mannobiose residues on the surface of target cells (e.g., cancer cells), thus increasing the sensitivity/strength of an immune response to the cancer being treated.

As such, in some embodiments, a subject method is a method of treating an individual having cancer by stimulating Dectin-2 signaling in myeloid cells, e.g., by inducing Dectin-2 clustering on the cell surface, thereby stimulating an anti-cancer immune response in the individual, and the method includes administering to the individual a composition that includes a subject Dectin-2 stimulating agent (e.g., a direct Dectin-2 stimulating agent such as a composition that includes a Dectin-2 binding glycopolymer, e.g., oligomannose glycopolypeptide, and/or a Dectin-2 antibody; or an indirect Dectin-2 stimulating agent such as an alpha-mannosidase class I inhibitor, a gene editing agent that reduces mannosidase expression and/or activity, an RNAi agent that can reduce mannosidase expression and/or activity, and the like).

In some embodiments, a method of treating an individual having cancer by stimulating Dectin-2 signaling in myeloid cells (e.g., by increasing Dectin-2 density on the cell surface, e.g., by inducing Dectin-2 clustering on the cell surface) thereby stimulating an anti-cancer immune response in the individual, and the method includes administering to the individual a Dectin-2 stimulating composition comprising one or more Dectin-2 stimulating agents selected from: (a) a non-plant derived naturally existing ligand for Dectin-2 (e.g., a mannan polysacharide, a mannan extract such as an extract from S. cerevisiae, a fungal cell wall extract such as an a cell wall extract from M. furfur and/or C. albicans); (b) a synthetic Dectin-2 stimulating glycopolymer or mimetic thereof (e.g., a glycopolypeptide, e.g., oligomannose glycopolypeptide such as a mannobiose-rich glycoprotein, e.g., an O-linked and/or N-linked mannobiose-rich glycoprotein); (c) a Dectin-2 stimulating anti-Dectin-2 antibody (e.g., an anti-Dectin-2 antibody—in some cases soluble and in some cases immobilized on a solid support, a multivalent anti-Dectin-2 antibody that also binds specifically to a cancer antigen); and (d) an alpha-mannosidase class 1 inhibitor (e.g., where cancer cells are contacted with the inhibitor an thereby increase the levels of Dectin-2 ligands on their surface).

### (a) Non-Plant Derived Naturally Existing Ligand for Dectin-2

In some embodiments, a method of treating an individual having cancer includes administering to the individual a composition comprising a “naturally existing ligand for Dectin-2”. Such a term encompasses the non-plant derived naturally existing ligands for Dectin-2 discussed below, but would also ecompass plant derived Dectin-2 stimulating ligands such as oligomannose glycopolypeptides, oligomannose/high-mannose glycans, mannobiose-rich glycoproteins, O-linked and/or N-linked mannobiose-rich glycoproteins, and mannan polysaccharides, obtained from natural sources (e.g., soybean agglutinin).

In some embodiments, a method of treating an individual having cancer includes administering to the individual a composition comprising a non-plant derived naturally existing ligand for Dectin-2 (i.e., a non-plant derived naturally existing Dectin-2 stimulating agent). In some cases, a subject composition comprising a non-plant derived naturally existing ligand for Dectin-2 includes a fungal cell wall extract that includes one or more glycoproteins that stimulate Dectin-2 signaling in myeloid cells (e.g., by increasing Dectin-2 density on the cell surface, e.g., by inducing Dectin-2 clustering on the cell surface). In some cases, such a fungal cell wall extract includes a Dectin-2 stimulating glycopolymer or mimetic thereof (e.g., a glycopolypeptide) (e.g., a Dectin-2 stimulating oligomannose glycopolypeptide such as a mannobiose-rich glycoprotein, e.g., an O-linked and/or N-linked mannobiose-rich glycoprotein). For example, see Ishikawa et al., Cell Host Microbe, 2013 Apr. 17; 13(4):477-88.

In some cases, a subject composition comprising a non-plant derived naturally existing ligand for Dectin-2 includes mannan polysaccharide (e.g., S. cerevisiae mannan such as an alkaline mannan extract, e.g., see product m7504 of Sigma-Aldrich, or C. albicans mannan). For example, see Uryu et al., Blood, 2015 May 7; 125(19):3014-23; and Saijo et al., Immunity, 2010 May 28; 32(5):681-91. A composition comprising a non-plant derived naturally existing ligand for Dectin-2 can include oligomannose/high-mannose glycans (manno-oligosaccharide) obtained from a natural source (e.g. man-9 from porcine thyroglobulin, and the like). In some cases, it is not obtained from a natural source, but is instead a laboratory generated product (e.g., is synthesized) but is structually different than a plant-derived natural product. A composition comprising a non-plant derived naturally existing ligand for Dectin-2 can include oligomannose glycans such as can be found in mannan extract from S. cerevisiae. In some cases mannan (e.g., a mannan polysaccharide, a composition comprising a oligomannose/high-mannose glycan) is delivered systemically to an individual (e.g., a human) and can treat multiple tumor types (e.g., cancers such as pancreatic, lung, and colon cancer). See, e.g., the Examples section below.

As used herein, the term “a composition that includes a Dectin-2 binding oligomannose glycopolypeptide” is meant to encompass a composition comprising a non-plant derived naturally existing ligand for Dectin-2 (e.g., a Dectin-2 stimulating cell wall extract such as an Malassezia furfur (M. furfur) Dectin-2 stimulating cell wall extract, for example such an extract that includes a Dectin-2 stimulating oligomannose glycopolypeptide such as a mannobiose-rich glycoprotein, e.g., an O-linked and/or N-linked mannobiose-rich glycoprotein). Likewise, the phrase “a glycopolypeptide that binds to Dectin-2” is meant to encompass the Dectin-2 stimulating glycopolypeptide(s) that can be found in a cell wall extract from M. furfur (e.g., an oligomannose glycoprotein, e.g., a mannobiose-rich glycoprotein, e.g., an O-linked and/or N-linked mannobiose-rich glycoprotein).

In some cases, a subject composition that includes a non-plant derived naturally existing ligand includes one or more glycoproteins (e.g., oligomannose glycoproteins such as mannobiose-rich glycoproteins, e.g., O-linked and/or N-linked mannobiose-rich glycoproteins), (in some cases isolated from a fungal cell wall extract), that independently or in combination stimulate Dectin-2 signaling. In some cases, a cell wall extract is an M. furfur cell wall extract. In some cases, the cell wall extract is from an M. furfur and/or C. albicans. In some cases, a subject composition comprising a non-plant derived naturally existing ligand comprises an extract from one or more of: M. furfur, C. albicans, Schistosoma mansoni, Mycobacterium tuberculosis, and Dermatophagoides farina, wherein the extract comprises one or more glycoproteins that stimulate Dectin-2 signaling.

A cell wall extract can be made using any convenient method. For example, see Ishikawa et al., Cell Host Microbe. 2013 Apr. 17; 13(4):477-88; and McGreal et al., Glycobiology. 2006 May; 16(5):422-30, which references are hereby incorporated by reference in their entirety. In some cases, a suitable cell wall extract is commercially available. For example, in some cases a suitable cell wall extract is a commercially available cell wall extract of M. furfur (e.g., furfurman; Invivogen).

In some cases, a method of treating an individual with cancer includes administering to the individual a composition that includes a non-plant derived naturally existing ligand for Dectin-2 (e.g., a fungal cell wall extract from M. furfur; mannan, e.g., a mannan polysaccharide, a composition comprising a oligomannose/high-mannose glycan; mannan from S. cerevisiae; and the like), In some cases, a method of treating an individual with cancer includes administering to the individual a composition that includes a non-plant derived naturally existing Dectin-2 stimulating glycoprotein (e.g., an oligomannose glycopolypeptide such as a mannobiose-rich glycoprotein, e.g., an O-linked and/or N-linked mannobiose-rich glycoprotein, e.g., such as can be found in a fungal cell wall extract from M. furfur). In some cases, a method of treating an individual with cancer includes administering to the individual a composition that includes a non-plant-derived high-mannose glycoconjugate (glycopolymer) such as a glycolipid or N-glycopeptide or protein fragment.

### (b) Synthetic Glycopolymers (e.g., Glycopolypeptides, e.g., Oligomannose Glycopolypeptides)

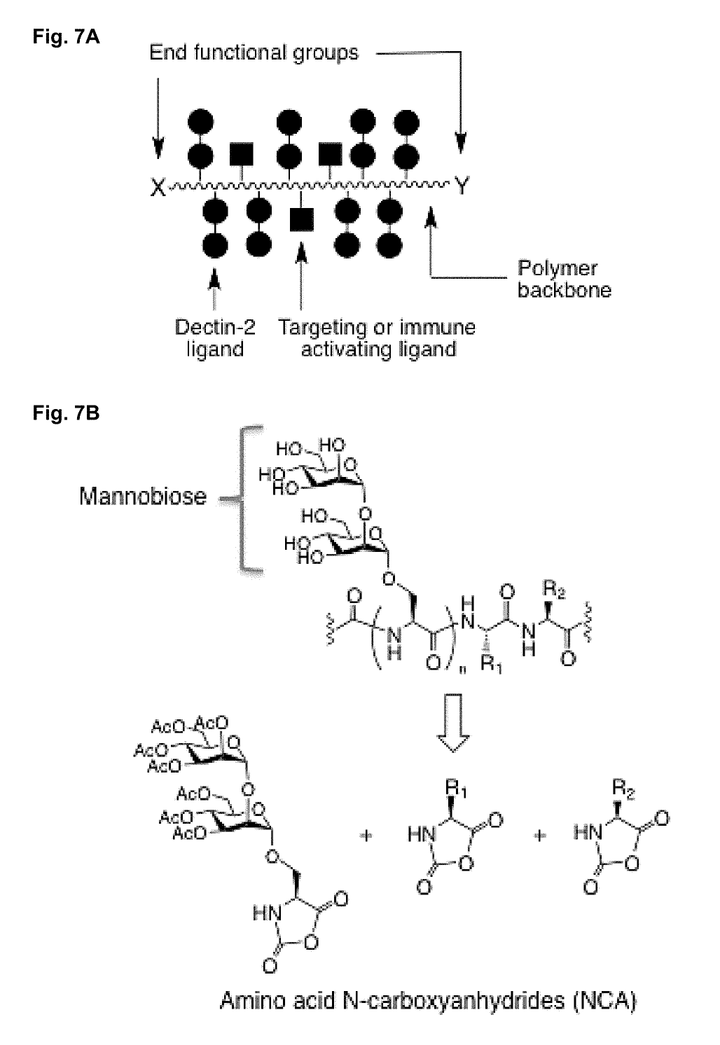

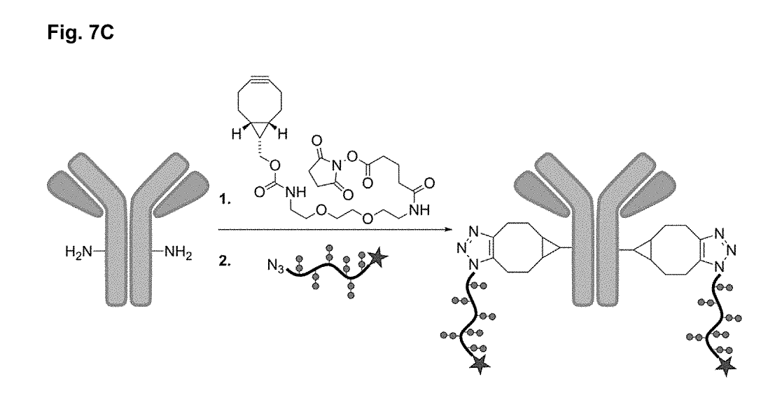

Densely O-glycosylated Serine-rich proteins adopt extended rigid structures with glycans displayed in a specific geometrical arrangement (e.g., that in some cases constitute a discrete pattern recognized by Dectin-2). This structure can be emulated with chemically defined glycopolymers tailored for potent Dectin-2 activation and functionalized for additional properties such as tumor targeting and immune cell activation (FIG. 7A). Synthetic glycopolymers can be designed with variable backbone structures, glycan structures, lengths, rigidities, geometries and end-functionalities. For example, Dectin-2 activating glycopolymers can include polypeptide backbones generated by polymerization of amino acid N-carboxyanhydrides (NCAs) (e.g., see FIG. 7B). The glycopolypeptides include glycosylated amino acid building blocks, such as mannobiosyl-serine, alone or blended with other amino acids to achieve various glycan densities and patterns. For an example of synthesis methodologies, see, e.g., Kramer et al., Proc Natl Acad Sci USA. 2015 Oct. 13; 112(41):12574-9, which is hereby incorporated by reference in its entirety.

Synthetic Dectin-2 stimulating glycopolypepeptides as used herein can include a mannobiose-modified serine. In some cases, a synthetic Dectin-2 stimulating glycopolypeptide is a mucin-like glycoprotein bearing a high density of serine O-glycosylation with Manα1,2Man (mannobiose), e.g., like the natural glycopolypeptide from M. furfur extracts, and acts as potent Dectin-2 agonist. In some cases, a subject synthetic glycopolypeptide activates Dectin-2 only when immobilized. In some cases, a subject synthetic glycopolypeptide activates Dectin-2 only when in soluble form. In some cases, a subject synthetic glycopolypeptide activates Dectin-2 when immobilized or when in soluble form. A subject synthetic glycopolypepeptide can be used like the natural ligand, e.g., as described above, or be can incorporated into a multivalent Dectin-2 stimulating agent (e.g., a tumor-targeting glycoconjugate (glycopolymer), described in more detail below). For an example of one possible conjugation strategy (that was used successfully) that can be used to conjugate a synthetic glycopeptide to an antibody, see FIG. 7C (e.g., Lysine residues on the antibody can be treated with NHS-cyclooctyne compounds, followed by bioorthogonal covalent reaction with azide terminal glycopeptides).

In addition, blended amino acid building blocks can be functionalized with moieties for tissue targeting, imaging, alternative immune activating ligands, and other desired functionalities. The synthetic approach allows for variation of glycan structure, density and intervening functionalities so that optimal constructs for Dectin-2 activation can be identified. Other properties related to therapeutic development can also be modulated. Glycopolymer functionalities can include imaging probes, groups for attachment to antibodies or integration into liposomes, or other desirable elements. In some cases, a subject synthetic Dectin-2 stimulating glycopolypeptide includes a peptide (e.g., a mucin-like peptide)(e.g., in some cases a peptide immobilized on a solid support, in some cases a soluble peptide, in some cases a peptide conjugated to a tumor-targeting moiety such as an antibody, etc.) having a length in the range of from 8 to 400 amino acids (e.g., 8 to 350, 8 to 300, 8 to 250, 8 to 200, 8 to 150, 8 to 125, 8 to 100, 8 to 75, 8 to 50, 8 to 35, 8 to 25, 8 to 20, 8 to 15, 10 to 400, 10 to 350, 10 to 300, 10 to 250, 10 to 200, 10 to 150, 10 to 125, 10 to 100, 10 to 75, 10 to 50, 10 to 35, 10 to 25, 20 to 400, 20 to 350, 20 to 300, 20 to 250, 20 to 200, 20 to 150, 20 to 125, 20 to 100, 20 to 75, 20 to 50, 20 to 35, 20 to 25, 35 to 400, 35 to 350, 35 to 300, 35 to 250, 35 to 200, 35 to 150, 35 to 125, 35 to 100, 35 to 75, 35 to 50, 50 to 400, 50 to 350, 50 to 300, 50 to 250, 50 to 200, 50 to 150, 50 to 125, 50 to 100, 50 to 75, 75 to 400, 75 to 350, 75 to 300, 75 to 250, 75 to 200, 75 to 150, 75 to 125, 75 to 100, 100 to 400, 100 to 350, 100 to 300, 100 to 250, 100 to 200, 100 to 150, or 125 to 400, 125 to 350, 125 to 300, 125 to 250, 125 to 200, or 125 to 150 amino acids). In some cases, a subject synthetic Dectin-2 stimulating glycopolypeptide includes a peptide (e.g., a mucin-like peptide) having a length in the range of from 50 to 150 amino acids (e.g., 50 to 125, 50 to 100, 50 to 75, 75 to 150, 75 to 125, 75 to 100, 100 to 150, or 125 to 150 amino acids).

Glycomimetic ligands for Dectin-2 can also be used in place of natural sugars in polymeric constructs. Glycomimetics can include non-hydrolyzable sugar analogs or non-sugar synthetic ligands that bind to and activate Dectin-2 (e.g., similarly to glycan ligands).

### (c) Dectin-2 Stimulating Anti-Dectin-2 Antibody (e.g., Immobilized on a Solid Support or Soluble)

In some embodiments, a method of treating an individual having cancer includes administering to the individual a composition comprising a Dectin-2 stimulating anti-Dectin-2 antibody (e.g., a humanized antibody, an antigen binding region of an anti-Dectin-2 antibody, and the like). In some cases, a subject Dectin-2 stimulating agent is an anti-Dectin-2 monospecific, multivalent antibody. In some cases, the anti-Dectin-2 antibody (e.g., a monoclonal anti-Dectin-2 antibody) is soluble (not immobilized on a solid support). In some cases, the anti-Dectin-2 antibody is immobilized on a solid support (e.g., a nanoparticle). Any convenient solid support can be used. Suitable solid supports include but are not limited to: plates, tubes, beads (glass or polystyrene beads), nylon, nitrocellulose, cellulose acetate, glass fiber, any convenient porous polymer, a colloidal particle, metallic nanomaterial such as nanoparticle, nanoplate, or nanoshell, a latex bead, etc. In some embodiments the solid support is a pharmaceutically acceptable solid support (e.g., a nanoparticle approved for therapeutic use).

In some cases, a subject Dectin-2 stimulating antibody is immobilized on a solid support that is a nanocarrier. Examples of nanocarriers for delivery of a subject Dectin-2 stimulating agent include but are not limited to: (a) polymeric nanoparticles in which drugs are conjugated to or encapsulated in polymers; (b) polymeric micelles: amphiphilic block copolymers that form to nanosized core/shell structure in aqueous solution (The hydrophobic core region serves as a reservoir for hydrophobic drugs, whereas hydrophilic shell region stabilizes the hydrophobic core and renders the polymer to be water-soluble); (c) dendrimers: synthetic polymeric macromolecule of nanometer dimensions, which is composed of multiple highly branched monomers that emerge radially from the central core; (d) liposomes: self-assembling structures composed of lipid bilayers in which an aqueous volume is entirely enclosed by a membranous lipid bilayer; (e) viral-based nanoparticles: in general structure are the protein cages, which are multivalent, self-assembles structures; and (f) carbon nanotubes: carbon cylinders composed of benzene rings.

Microparticles (e.g., beads) can serve as solid supports or substrates to which other materials, such as a subject anti-Dectin-2 antibody, can be coupled/conjugated. A range of bead sizes can be used depending on the nature of use (e.g., contacting a cell such as a myeloid or cancer cell in vitro versus administration into an individual). For example, a solid support bead can range in size from 0.01 to 1,000 μm (e.g., 0.1 to 100 μm, 1 to 100 μm, 1 to 10 μm, etc.) in diameter. In some embodiments, the beads can range in size from 2.5 to 3 μm (e.g., 2.7 to 2.9 μm, 2.5 μm, 2.6 μm, 2.7 μm, 2.8 μm, 2.9 μm, or 3.0 μm) in diameter. In some cases, it may be advantageous to use larger beads. In some embodiments, the beads can range in size from 4.3 to 5.5 μm in diameter (e.g., 4.4-4.6 μm, 4.3 μm, 4.4 μm, 4.5 μm, 4.6 μm, 4.7 μm, 4.9-5.1 μm, 4.9 μm, 5.0 μm, 5.1 μm, 5.2 μm, 5.3 μm, 5.4 μm, or 5.5 μm in diameter). In some embodiments, a solid support bead can have a size in a range of from 2 to 15 μm in diameter (e.g., 2 to 12 μm, 2 to 10 μm, 2 to 8 μm, 2 to 6 μm, 2 to 5 μm, 2 to 4 μm, 3 to 15 μm, 3 to 12 μm, 3 to 10 μm, 3 to 8 μm, 3 to 6 μm, 3 to 5 μm, 3 to 4 μm, 4 to 15 μm, 4 to 12 μm, 4 to 10 μm, 4 to 8 μm, 4 to 6 μm, 4 to 5 μm, 5 to 15 μm, 5 to 12 μm, 5 to 10 μm, 5 to 8 μm, 5 to 6 μm in diameter).

Subject beads can be made of any convenient material (or combinations thereof), including, but not limited to inorganics such as metals, silica (e.g., Si0₂), glass, alumina, titania, ceramic, etc.; organics such as polystyrene, polymethylmethacrylate (PMMA); melamine, polyactide, etc.; and magnetic materials such as silica, polystyrene, dextran, etc. Commercially available magnetic beads include but are not limited to ProMag, COMPEL, BioMag, BioMagPlus, and Dynabeads. Microparticles in a variety of sizes and polymer compositions that are suitable for use in the preparation of a subject anti-Dectin-2 antibody immobilized on a solid support. Microparticles can also be stained, e.g., with a fluorescent dye. Compositions of, and methods of producing, suitable beads can be found in both the patent and non-patent scientific literature (e.g., U.S. Pat. Nos. 8,283,037; 5,597,531; 5,635,574; and 8,163,183, which are incorporated herein by reference).

Multivalent, monospecific anti-Dectin-2 antibodies are also envisioned. Various methods for generating multivalent, monospecific antibodies have been described and can be used to generate Dectin-2-specific antibody complexes.

### Multivalent Dectin-2 Stimulating Agents

In some embodiments a Dectin-2 stimulating agent is multivalent (e.g., multifunctional). For example, in some cases, a direct Dectin-2 stimulating agent (the ‘first agent’) is conjugated to a ‘second agent’. The first agent can be any direct Dectin-2 stimulating agent (i.e., an agent that binds to Dectin-2 and stimulates Dectin-2 signaling in myeloid cells, e.g., an antigen binding region of an anti-Dectin-2 antibody, a glycopolymer such as a glycopolypeptide that binds to Dectin-2, a natural Dectin-2 ligand such as mannobiose-rich glycoprotein or mannan, etc.). Thus, in some cases the first agent of a multivalent Dectin-2 stimulating agent is an anti-Dectin-2 antibody or an antigen binding region of an anti-Dectin-2 antibody. In some cases the first agent of a multivalent Dectin-2 stimulating agent is a glycopolymer such as a glycopolypeptide that binds to Dectin-2. In some cases the first agent of a multivalent Dectin-2 stimulating agent is a natural Dectin-2 ligand (e.g, a non-plant derived naturally existing ligand for Dectin-2, e.g., a fungal cell wall extract such as one from Malassezia furfur (M. furfur) and/or Candida albicans; mannan, e.g., a mannan extract such as an alkaline extract, e.g., a mannan extract from S. cerevisiae; and the like).

In some cases, the second agent of a multivalent Dectin-2 stimulating agent is an immunomodulatory agent (e.g., a cytokine, a growth factor, a stimulatory ligand for a pattern recognition receptor (PRR), etc.). For example, in some cases the second agent is a cytokine. Examples of cytokines include, but are not limited to IL-I, IL-2, IL-3, IL-4, IL-6, IL-7, IL-9, IL-10, IL-12, IL-15, IL-18, IL-21, IFN-α, IFN-β, IFN γ, G-CSF, TNFα, and GM-CSF. Thus in some cases a second agent is selected from: IL-I, IL-2, IL-3, IL-4, IL-6, IL-7, IL-9, IL-10, IL-12, IL-15, IL-18, IL-21, IFN-α, IFN-β, IFN γ, G-CSF, TNFα, and GM-CSF. In some cases the second agent is GM-CSF. In some cases, the second agent is a cytokine such as interferon gamma (IFNγ), IL-15, IFN-α, or IFN-β.

In some cases the second agent of a multivalent Dectin-2 stimulating agent is a growth factor. Examples of growth factors include, but are not limited to colony stimulating factor (CSF), Ativin, Connective Tissue Growth Factor (CTGF), Epidermal Growth Factor (EGF), Erythropoietin, Fibroblast Growth Factor (FGF), Galectin, Growth Hormone, Hepatoma-Derived Growth Factor (HDGF), Hepatocyte Growth Factor (HGF), an Insulin-Like Growth Factor Binding Protein (IGFBP-1, -3, -4, -5, -6, 7, and the like), Insulin, Insulin-Like Growth Factor (e.g., IGF-1, -2, -3), Keratinocyte Growth Factor (KGF), Leptin, Macrophage Migration Inhibitory Factor (MIF), Melanoma Inhibitory Activity (MIA), Myostatin, Noggin, Omentin, Oncostatin-M, Osteopontin, Osteoprotegerin, Platelet-Derived Growth Factor (PDGF), Periostin, Placenta Growth Factor (PLGF), Placental Lactogen, Prolactin, RANK Ligand (RANKL), Retinol Binding Protein (RBP), Stem Cell Factor (SCF), Transforming Growth Factor (TGFβ), and Vascular Endothelial Growth Factor (VEGF). In some cases the second agent is a factor that induces Dectin-2 expression. In some cases the second agent is GM-CSF.

In some cases the second agent of a multivalent Dectin-2 stimulating agent is a stimulatory ligand for a pattern recognition receptor (PRR). Examples of PRRs include, but are not limited to Toll-like receptors (TLRs)(e.g., TLR7/8), nucleotide-binding oligomerization domain-like receptors (NLRs), C-type lectin receptors (CLRs), and RIG-I-like receptors (RLRs). Thus in some cases a second agent is selected from: a Toll-like receptor (TLR), nucleotide-binding oligomerization domain-like receptor (NLR), C-type lectin receptor (CLR), and RIG-I-like receptor (RLR)

Examples of immunomodulator agents (which can be used as the second agent of a multivalent Dectin-2 stimulating agent) include but are not limited to: an anti-CTLA4 antibody (or antigen-binding region thereof); an anti-PD-1/PD-L1 agent (e.g., an anti-PD-1 antibody or antigen-binding region thereof, a PD-1-binding reagent such as a PD-L1 or PD-L2 ectodomain, an anti-PD-L1 antibody or antigen-binding region thereof, a PD-L1-binding reagent such as a PD-1 ectodomain, and the like); a CD40 agonist (e.g., CD40L or anti-CD40 antibody); a 4-1BB modulator (e.g., a 4-1BB-agonist); an anti-CD47/SIRPA agent (e.g., an anti-CD47 antibody or antigen-binding region thereof, a CD47-binding reagent such as a SIRPA ectodomain, an anti-SIRPA antibody or antigen-binding region thereof, a SIRPA-binding reagent such as a CD47 ectodomain, and the like); an inhibitor of TIM3 and/or CEACAM1; an inhibitor of TIM3 and/or CEACAM1; an inhibitor of BTLA and/or CD160; and the like. Thus, an immunomodulator agent can be a checkpoint blockade agent.

As one illustrative example, in some cases the first agent of a multivalent Dectin-2 stimulating agent is a Dectin-2-binding glycopolymer (or an anti-Dectin-2 antibody, or a natural Dectin-2 ligand such as mannan) and the second agent is granulocyte-macrophage colony-stimulating factor (GM-CSF) (i.e., the multivalent Dectin-2 stimulating agent is a first agent conjugated to GM-CSF).

In some cases, a direct Dectin-2 stimulating agent (i.e., an agent that binds to Dectin-2 and stimulates Dectin-2 signaling in myeloid cells, e.g., an antigen binding region of an anti-Dectin-2 antibody, a glycopolymer such as a glycopolypeptide that binds to Dectin-2, a natural Dectin-2 ligand, etc.) is conjugated to a targeting agent that targets the Dectin-2 stimulating agent to a target cell (e.g., a cancer cell) such that the Dectin-2 stimulating agent is displayed on the surface of the target cell. Thus, in some cases the second agent of a multivalent Dectin-2 stimulating agent includes a targeting agent (e.g., an antigen binding portion of a tumor-antigen antibody) that targets the Dectin-2 stimulating agent to a target cell (e.g., a cancer cell). Because such an agent can bind to two different target molecules (e.g., a target molecule such as a cancer antigen on the surface of a cancer cell, and Dectin-2 on the surface of a myeloid cell), a Dectin-2 stimulating agent that is conjugated to a targeting agent is sometimes referred to herein as a multivalent Dectin-2 stimulating agent.

In some embodiments, a subject Dectin-2 stimulating agent (e.g., for treating cancer via Dectin-2 stimulation) is a multivalent agent (e.g., a multivalent antibody, an antibody-glycoconjugate, and the like) that includes (i) a first agent: a Dectin-2 stimulating agent that binds, e.g., specifically binds, to Dectin-2 on the surface of a myeloid cell and stimulates Dectin-2 signaling (i.e., a direct Dectin-2 stimulating agent); and (ii) a second agent: a cancer targeting agent (e.g., (a) a cancer cell targeting agent, i.e., a targeting agent that specifically binds to a cancer antigen (e.g., an antitumor antibody, a tumor-binding peptide, a tumor-binding aptamer, and the like); and/or (b) an immunomodulator agent, an agent that specifically binds to a cancer immunotherapy target).

An example of a targeting agent that specifically binds to a cancer antigen (and can be used as a second agent of a multivalent Dectin-2 stimulating agent) is a binding region of an antibody that specifically binds to a cancer cell antigen. Suitable cancer antigens are those antigens that are associated with cancer. Examples of cancer antigens (e.g., to which a targeting agent can specifically bind) include, but are not limited to: CD19, CD20, CD22, CD24, CD25, CD30, CD33, CD38, CD44, CD47, CD52, CD56, CD70, CD96, CD97, CD99, CD123, CD279 (PD-1), CD274 (PD-L1), EpCam, EGFR, 17-1A, HER2, CD117, C-Met, PTHR2, HAVCR2 (TIM3), and SIRPA. Additional examples of targeting agents that specifically binds to a cancer antigen (and can be used as a second agent of a multivalent Dectin-2 stimulating agent) include but are not limited to tumor-binding peptides and tumor-binding aptamers.

In some cases, a subject multivalent Dectin-2 stimulating agent (e.g., for treating cancer via Dectin-2 stimulation) includes (i) a glycopolymer such as a glycopolypeptide (e.g., a naturally occurring or synthetic glycopolypeptide such as an oligomannose glycopolypeptide, e.g., as described above), which serves to stimulate Dectin-2 signaling in myeloid cells; and (ii) a targeting agent such as a cancer cell targeting agent (e.g., an antigen binding region of an antibody against a cancer antigen, a tumor-binding peptide, a tumor-binding aptamer). In some cases, a subject multivalent Dectin-2 stimulating agent (e.g., for treating cancer via Dectin-2 stimulation) includes (i) a glycopolymer such as a glycopolypeptide (e.g., a naturally occurring or synthetic glycopolypeptide such as an oligomannose glycopolypeptide, e.g., as described above), which serves to stimulate Dectin-2 signaling in myeloid cells; and (ii) an immunomodulatory agent (e.g., an anti-cancer binding region of an antibody against a checkpoint inhibitor, a CD40 agonist such as CD40L or anti-CD40 antibody, a T-cell regulated co-stimulatory molecule, a checkpoint blockade agent, a polypeptide that specifically binds to a cancer target, e.g., an ectodomain that binds to a cancer antigen, an ectodomain that specifically binds to a cancer immunotherapy target such as PD-1, PD-1L, CD47, SIRPA, CTLA4, and the like).

Glycoconjugates (e.g., oligomannose glycopolypeptides) with anti-cancer targeting elements (e.g., tumor-targeting elements) can spare normal tissues and lead to the selective display of Dectin-2 ligands (e.g., Dectin-2 binding region from an anti-Dectin-2 antibody, an oligomannose glycopolypeptide, mannan polysaccharide or other oligomannose glycans, etc.) on tumor cells. The flexibility of these engineered products also presents opportunities for functional optimization and for tailoring of the products to specific cancers. For example, in some cases a subject multivalent Dectin-2 stimulating agent includes an antigen recognition region from an anti-tumor antibody conjugated to a synthetic or natural Dectin-2 ligand. In some cases, anti-Dectin-2 antibodies are used as multivalent complexes of Dectin-2 antibodies or ligands (e.g. mannobiose-rich glycopeptides and/or other oligomannose glycans such as Man-9) that resemble microbes with a high density of Dectin-2 ligands, like M. furfur (e.g., by immobilizing an anti-Dectin-2 antibody on a solid support). For an example of one possible conjugation strategy (that was used successfully) that can be used to conjugate a synthetic glycopeptide to an antibody, see FIG. 7C (e.g., Lysine residues on the antibody can be treated with NHS-cyclooctyne compounds, followed by bioorthogonal covalent reaction with azide terminal glycopeptides).

Bispecific, multivalent antibodies can include antigen recognition domains (Fab, scFv, scDb, etc.) for both Dectin-2 and a tumor-associated cell surface molecule (e.g. CD19, CD20, CD22, CD24, CD25, CD30, CD33, CD38, CD44, CD47, CD52, CD56, CD70, CD96, CD97, CD99, CD123, CD279 (PD-1), CD274 (PD-L1), EpCam, EGFR, 17-1A, HER2, CD117, C-Met, PTHR2, HAVCR2 (TIM3), and the like). These engineered antibodies could resemble other iterations of bispecific antibodies targeting both immune cells and tumor cells, like the bivalent bispecific T cell engagers (BITE) or tetravalent bispecific antibodies (TandAb) undergoing clinical development. The glycoconjugates can include a tumor-targeting component (e.g. an antibody against a cancer antigen such as an EpCAM antibody, a tumor-binding peptide, a tumor-binding aptamer, and the like) combined with glycans recognized by Dectin-2 (e.g. oligomannose glycopeptides, mannan polysaccharide, and/or other oligomannose glycans such as Man-9) (e.g. a subject Dectin-2 stimulating glycopolypeptide). In some cases, the tumor-targeting component can be directly modified to display multiple copies of a Dectin-2 ligand, or a linker (e.g. biotin) to a glycan-modified protein (e.g. streptavidin) may be used to increase glycan valency and avoid any disruption of tumor antigen-binding resulting from carbohydrate modification. Any convenient method for modifying proteins with carbohydrates can be use, e.g., protocols for conjugation of complex carbohydrates such as oligomannose glycans (e.g., see Gildersleeve et al, Bioconjug Chem. 2008 July; 19(7):1485-90). Tumor cells covered with such molecules would thus resemble Dectin-2 ligand-expressing microbes and, like kifunensine-treated tumor cells, activate Dectin-2 signaling at points of contact between TAM cells and tumor cells.

Both the Dectin-2-activating and tumor-targeting domains may be modified with regard to specificity, stability, affinity, and valency or be combined with other molecules (e.g. other PRR ligands, cytokines, toxins, etc.). For example, antibody-based glycoconjugates may be modified to display a very high density of oligomannose glycans (e.g., Man-9) to more efficiently trigger Dectin-2 signaling. Meanwhile, the variable region of the antibody component may be changed to recognize specific tumor antigens, and the constant region modified (e.g. by antibody class switching or afucosylation) to more effectively engage activating Fc receptors on TAM cells. Simultaneously activating both Dectin-2 and Fc receptor signaling in this way can lead to more efficient tumor cell killing, uptake, and antigen presentation and, subsequently, more robust adaptive immune responses.

Various methods for generating multivalent antibodies have been described and can be used to generate Dectin-2-specific antibody complexes. Similarly, the production of carbohydrate-based compounds have been described (e.g. glycopolymers, glycodendrimers, glycoclusters, glyconanoparticles), including several that incorporate glycans recognized by Dectin-2. Both these antibody- and carbohydrate-based complexes can be used to directly stimulate TAM in a Dectin-2-dependent fashion (i.e., as Dectin-2 stimulating agents), similar to the cell wall extract from M. furfur.

### (d) Alpha-Mannosidase Class 1 (α-Mannosidase I) Inhibitor

In some embodiments, a method of treating an individual having cancer includes administering to the individual a composition comprising an alpha-mannosidase class 1 inhibitor (e.g., kifunensine; 1-deoxymannojirimycin; an RNAi agent that specifically reduces the expression of one or more mannosidases selected from: MAN1B1, MAN1A1, MAN1A2, and MAN1C1; a gene editing agent that specifically reduces the expression of one or more mannosidases selected from: MAN1B1, MAN1A1, MAN1A2, and MAN1C1). Dectin-2 recognizes various pathogen components containing multiple terminal mannose residues and reacts strongly with high-mannose type glycans. High-mannose glycans are common intermediate glycan species generated during N-linked glycosylation of proteins in eukaryotic cells. In mammalian cells, these high-mannose glycans are further processed into complex or hybrid type N-glycans—a process which requires the action of various mannosidases that cleave terminal mannose residues from the initial high-mannose precursor, Man₉GlcNAc₂ (Man-9). Consequently, treating cells with a mannosidase inhibitor (e.g. kifunensine and/or 1-deoxymannojirimycin, which are alpha-mannosidase class I inhibitors) leads to a shift in N-linked glycosylation patterns, resulting in the production of glycoproteins predominantly modified with high-mannose species (e.g. Man-9), and therefore resulting in cells that display increased levels of Dectin-2 ligands on their surface.

Small molecule alpha-mannosidase class I inhibitors (also referred to as alpha-mannosidase I inhibitors) (i.e. kifunensine, 1-deoxymannojirimycin, and the like) act upon multiple mannosidases in the endoplasmic reticulum (ER) and Golgi complex of mammalian cells. In some cases, a subject alpha-mannosidase class I inhibitor is a selective inhibitor of ER α-mannosidase I (MAN1B1), which is the first mannosidase to act upon Man-9, the highest order high-mannose species in the N-glycosylation pathway. However, Dectin-2 also binds to lower order high-mannose glycans (e.g. Man-7/-8). Thus, in some cases, a subject alpha-mannosidase class I inhibitor is a selective inhibitor of one or more downstream golgi mannosidases (MAN1A1, MAN1A2, MAN1C1).

In some cases, the inhibitor is a small molecule (e.g., an α-mannosidase class I inhibitor such as kifunensine and/or 1-deoxymannojirimycin). In some cases, the inhibitor (also referred to herein as an alpha-mannosidase class 1 reduction agent) is an RNAi agent or a gene editing agent that targets a mannosidase (e.g., reduces the expression of a one or more mannosidases selected from: MAN1B1, MAN1A1, MAN1A2, MAN1C1). The genes that can be targeted by an alpha-mannosidase class 1 reduction agent such as an RNAi agent or gene editing agent include: MAN1B1, MAN1A1, MAN1A2, and MAN1C1.

RNAi agents include shRNA, siRNA, and microRNA agents that specifically target RNAs that encode one or more proteins selected from MAN1B1, MAN1A1, MAN1A2, MAN1C1. In some cases, the RNAi agent specifically targets an RNA that encodes MAN1B1.

Gene editing agents include agents that that can target the genome of a cell to modify expression of a gene. In some cases, a gene editing agent is a CRISPR/Cas agent (e.g., cas protein(s) plus one or more appropriate guide RNAs, e.g., Cas9 plus guide RNA, cpf1 plus guide RNA). In some cases, a gene editing agent is a zing finger nuclease agent. In some cases, a gene editing agent is a TALE or TALEN agent. The term ‘gene editing agent” as used herein encompasses gene editing agents that cleave the targeted DNA to induce mutation (e.g., via homologous directed repair or non-homologous end-joining), and also includes gene editing agents that can reduce expression in the absence of target cleavage (e.g., gene editing agents that are fused or conjugated to expression modulators such as transcriptional repressors or epigenetic modifiers that can dampen/reduce expression).

The term “alpha-mannosidase class 1 inhibitor”, as used herein also encompasses prodrugged forms of mannosidase inhibitors, such as those with tumor-specific enzyme-activated caging groups, which can be employed for selective tumor targeting. In addition, alpha-mannosidase class 1 inhibitors can be incorporated into antibody-drug conjugates for tumor delivery.

In some embodiments, a method of treating an individual having cancer includes contacting a cancer cell from the individual with an alpha-mannosidase class 1 inhibitor in vitro or ex vivo, and introducing the contacted cancer cell into the individual. Without being bound by theory, this works because alpha-mannosidase class 1 inhibitors cause increased levels of Dectin-2 stimulatory compounds on the surface of target cells (e.g., cancer cells) (e.g., by increasing the display and/or density of terminal mannose/mannobiose residues on the cell surface), making the target cells more likely to stimulate an immune response and/or causing the target cells to stimulate a more intense immune response than would otherwise be stimulated. In some cases, the contacted cancer cell is administered systemically to the individual. In some cases, the contacted cancer cell is administered locally (e.g., into a tumor of the individual, s.c., i.d., i.m., etc.).

In some embodiments, a method of treating an individual having cancer includes contacting a cancer cell from the individual with an alpha-mannosidase class 1 inhibitor in vivo (e.g., by administering the alpha-mannosidase class 1 inhibitor to the individual). In some cases, the alpha-mannosidase class 1 inhibitor is delivered systemically. In some cases, the alpha-mannosidase class 1 inhibitor is delivered locally (e.g., into a tumor of the individual, into a region in which a tumor was recently resected, and the like).

Stimulating a Myeloid Cell, an APC, and/or a T Cell

In some embodiments (e.g., when the method includes administering to the individual a Dectin-2 stimulating composition that includes a subject Dectin-2 stimulating agent) an endogenous myeloid cell (a myeloid cell present in the individual) (e.g., a tumor-associated myeloid (TAM) cell, a dendritic cell (DC), a tumor associated DC, an antigen presenting cell (APC), a tumor associated APC, and the like) is contacted in vivo with the administered composition. Thus, the method can be considered an in vivo method of treating an individual having cancer. For example, a Dectin-2 stimulating composition can be administered to an individual (e.g., systemically or locally, e.g., injected into or near a tumor, into or near a site of tumor resection, and the like) and endogenous myeloid cells are thereby contacted with the Dectin-2 stimulating composition. The stimulated myeloid cells can then mount an enhanced immune response to the cancer cells, e.g., stimulated APCs can be loaded (e.g., uptake of a target antigen by the APC, e.g., for presentation to a T cell) and can then contact endogenous T cells in vivo.

Aspects of the disclosure include compositions and methods for stimulating an antigen presenting cell (APC) (e.g., a dendritic cell (DC), a macrophage, a B cell). In some embodiments, such methods include: (a) contacting in vitro or ex vivo a cancer cell with an alpha-mannosidase class 1 inhibitor to produce an inhibitor-contacted cancer cell (e.g., one that has increased display and/or density of terminal mannose/mannobiose residues on the surface of the cell, and therefore has increased surface levels of Dectin-2 ligands); and (b) contacting an APC with the inhibitor-contacted cancer cell (e.g., which can stimulate the APC cell to ‘load’ with a cancer antigen, e.g., which can stimulate the APC to engulf the cancer cell). In some cases, the method further includes introducing the contacted APC into the individual (e.g., which can then contact cancer cells and contact T cells to stimulate/enhance the immune response to the cancer cells). In some cases, the method further includes after contacting an APC with the inhibitor-contacted cancer cell (e.g., to ‘load’ the APC), contacting a T cell with the contacted (e.g., ‘loaded’) APC, thereby stimulating the T cell. In some cases, the method further includes introducing the stimulated T cell into the individual. Any or all of the cells (e.g., cancer cell, APC, T cell) can be autologous to an individual being treated. For example, in some cases, the T cell (e.g., just the T cell) is autologous to an individual being treated. In some cases, the APC (e.g., just the APC) is autologous to an individual being treated. In some cases, the cancer cell (e.g., just the cancer cell) is autologous to an individual being treated. In some cases, the cancer cell and APC are autologous to an individual being treated. In some cases, the cancer cell and T cell are autologous to an individual being treated. In some cases, the APC and T cell are autologous to an individual being treated. In some cases, the cancer cell, the APC, and the T cell are autologous to an individual being treated. In some cases, a step of contacting a T cell (e.g. of an individual) is in vivo. In some cases, the step of contacting a T cell (e.g. of an individual) is in vitro.

In some embodiments, a T cell is contacted with a loaded APC, e.g., DC. During contact, the loaded APC, e.g., DC, presents antigens to the T cell to produce a contacted T cell, and the contacted T cell generates an immune response specific to the presented antigens. The T cells can be CD4+ T cells, CD8+ T cells, or a combination of CD4+ and CD8+ T cells.

Contacting a T cell with a loaded APC, e.g., DC, can be in vitro or in vivo. Thus, the phrase “contacting a T cell” encompasses both in vitro and in vivo contact. If the contact is in vivo, loaded APCs, e.g., DCs, can be administered to the individual and the APCs, e.g., DCs, then contact endogenous T cells of the individual to induce an immune response. Thus, a step of “contacting a T cell of an individual with a loaded APC”, e.g., “contacting a T cell of an individual with a loaded DC,” when performed in vivo, can in some cases be written: “introducing into an individual a loaded DC.” For example, in some cases, a subject method includes: (a) contacting in vitro an APC, e.g., DC, from an individual with: (i) a target antigen; and (ii) a subject Dectin-2 stimulating agent, at a dose and for a period of time effective for the uptake of the target antigen by the APC, e.g., DC, thereby producing a loaded APC, e.g., DC; and (b) introducing into the individual the loaded APC, e.g., DC. APCs (e.g., DCs) and T cells can be administered to the individual as described below for the “administering cells”.

In some cases, the subject methods can be performed in vivo. In some such cases, contact is in vivo, endogenous APC, e.g., DC, are loaded in vivo, and the loaded APC, e.g., DC, then contact T cells in vivo. Thus, the method can be carried out by in vivo administration (e.g., administration of a subject Dectin-2 stimulating agent). For example, endogenous APC, e.g., DC (e.g., TADC), can be loaded in vivo by administering to an individual a composition that includes a subject Dectin-2 stimulating agent.

If the contact is in vitro, an autologous T cell (e.g., a population of autologous T cells) from the individual can be contacted with a loaded APC, e.g., DC, to produce a contacted T cell (e.g., a population of contacted T cells). A T cell can be contacted with a loaded APC, e.g., DC, for a period of time sufficient to activate the T cell such that the T cell with induce an immune response when administered to the individual. T cells (either prior to or after contact with a loaded APC, e.g., DC) can be expanded in vitro and/or modified (e.g., genetically modified) prior to being administered to the individual.

In some cases, a T cell is contacted in vitro with a loaded APC, e.g., DC, for a period of time in a range of from 5 minutes to 24 hours (e.g., 5 minutes to 18 hours, 5 minutes to 12 hours, 5 minutes to 8 hours, 5 minutes to 6 hours, 5 minutes to 4 hours, 5 minutes to 2 hours, 5 minutes to 60 minutes, 5 minutes to 45 minutes, 5 minutes to 30 minutes, 15 minutes to 18 hours, 15 minutes to 12 hours, 15 minutes to 8 hours, 15 minutes to 6 hours, 15 minutes to 4 hours, 15 minutes to 2 hours, 15 minutes to 60 minutes, 15 minutes to 45 minutes, 15 minutes to 30 minutes, 20 minutes to 18 hours, 20 minutes to 12 hours, 20 minutes to 8 hours, 20 minutes to 6 hours, 20 minutes to 4 hours, 20 minutes to 2 hours, 20 minutes to 60 minutes, 20 minutes to 45 minutes, 30 minutes to 18 hours, 30 minutes to 12 hours, 30 minutes to 8 hours, 30 minutes to 6 hours, 30 minutes to 4 hours, 30 minutes to 2 hours, 30 minutes to 60 minutes, 30 minutes to 45 minutes, 45 minutes to 18 hours, 45 minutes to 12 hours, 45 minutes to 8 hours, 45 minutes to 6 hours, 45 minutes to 4 hours, 45 minutes to 2 hours, 45 minutes to 60 minutes, 1 hour to 18 hours, 1 hour to 12 hours, 1 hour to 8 hours, 1 hour to 6 hours, 1 hour to 4 hours, 1 hour to 2 hours, or 1 hour to 90 minutes).

In some cases, a population of T cells (e.g., 1×10² or more cells (e.g., 1×10³ or more cells, 1×10⁴ or more cells, 1×10⁵ or more cells, or 1×10⁶ or more cells)) is contacted in vitro with a loaded APC, e.g., DC (e.g., a population of loaded APCs, e.g., DCs; a population having loaded APCs, e.g., DCs; etc.). In some cases, a population of T cells (e.g., in a range of from 1×10² to 1×10¹⁰ cells (1×10² to 1×10⁸ cells, 1×10³ to 1×10⁷ cells, 1×10⁴ to 1×10⁶ cells, 5×10⁴ to 5×10⁵ cells, or 1×10⁵ cells)) is contacted in vitro with a loaded APC, e.g., DC (e.g., a population of loaded APCs, e.g., DCs; a population having loaded APCs, e.g., DCs; etc.). In some cases, a T cell (e.g., a population of T cells) is contacted with a cell population (e.g., 1×10² or more cells (e.g., 1×10³ or more cells, 1×10⁴ or more cells, 1×10⁵ or more cells, or 1×10⁶ or more cells)) having loaded APCs, e.g., DCs (e.g., a cell population of loaded APCs, e.g., DCs). In some cases, a T cell (e.g., a population of T cells) is contacted with a cell population (e.g., in a range of from 1×10² to 1×10¹⁰ cells (1×10² to 1×10⁸ cells, 1×10³ to 1×10⁷ cells, 1×10⁴ to 1×10⁶ cells, 5×10⁴ to 5×10⁵ cells, or 1×10⁵ cells)) having loaded APCs, e.g., DCs (e.g., a cell population of loaded APCs, e.g., DCs).

The contacted T cell (e.g., cells of a contacted T cell population) can be administered to the individual as described below for the “administering cells”.

Dendritic Cells.

A dendritic cell (DC) is a type of antigen-presenting cell of the mammalian immune system. The term “dendritic cell” as used herein refers to any member of a diverse population of morphologically similar cell types found in lymphoid or non-lymphoid tissues. These cells are characterized by their distinctive morphology and high levels of surface MHC-class II expression (Steinman, et al., Ann. Rev. Immunol. 9:271 (1991); hereby incorporated by reference for its description of such cells).

Dendritic cells are present in nearly all tissues such as the skin and the inner lining of the nose, lungs, liver, stomach, and intestines, as well as in bone marrow, blood, spleen, and lymph nodes. Once activated, DC migrate to the lymph nodes where they interact with T cells and B cells to initiate and shape the adaptive immune response. At certain development stages DC grow branched projections (the dendrites) that give the cells their name. Examples of dendritic cells include bone marrow-derived dendritic cells (BMDC), plasmacytoid dendritic cells, Langerhans cells, interdigitating cells, veiled cells, and dermal dendritic cells. In some cases, a DC expresses at least one marker selected from: CD11 (e.g., CD11a and/or CD11c), MHC-class II (for example, in the case of human, HLA-DR, HLA-DP and HLA-DQ), CD40, CD80 and CD86. In some cases, a DC is positive for HLA-DR and CD83, and negative for CD14. In general DC can be identified (e.g., the presence of DC can be verified) based on any or all of the markers: CD11c+; CD14−/low; CD80+; CD86++; MHC Class I++, MHC Class II+++; CD40++; CD83+/−; CCR7+/−. In some cases, the DC is CD11b⁺/Gr1^{neg}/CD11c⁺/MHCII⁺/CD64^{dull}. In some cases, the DC is CD11b^{neg}/CD11c^{hi}/MHCII⁺.

In some cases, the dendritic cell expresses a specific Ig Fc receptor. For example, a dendritic cell can express an Fc-γ receptor which recognizes IgG antibodies, or antibodies that contain an Fc region of an IgG. As another example, the dendritic cell can express an Fc-α receptor which recognizes IgA antibodies, or antibodies that contain an Fc region of an IgA. As yet another example, the dendritic cell can express an Fc-ε receptor which recognizes IgE antibodies, or antibodies that contain an Fc region of an IgE. In some cases, dendritic cells expressing a specific Fc receptor are obtained and loaded with an appropriate bridging molecule (e.g., allogeneic Ig of a class recognized by the dendritic cell Fc receptor).

In some embodiments, subject methods include a step of obtaining or isolating a DC (e.g., isolating enriched populations of DC). Techniques for the isolation, generation, and culture of DC will be known to one of ordinary skill in the art and any convenient technique can be used. In some cases, the DC are autologous to the individual who is being treated (i.e., are cells isolated from the individual or are cells derived from cells of the individual).

In some cases, CD34(+) progenitors (e.g., bone marrow (BM) progenitor cells) are used as a source for generating DCs (e.g., CD34⁺ cells can be enriched using, for example, antibody-bound magnetic beads), which are then referred to as bone marrow (BM) derived dendritic cells (BMDC). For example, BMDCs can be generated by culturing nonadherent cells (CD34+ cells) in the presence of a cytokine that functions as a white blood cell growth factor (e.g., granulocyte-macrophage-colony stimulating factor (GM-CSF), e.g., 50 ng/ml) and a cytokine (e.g., interleukin 4 (IL-4), e.g., 20 ng/ml). In some cases, the CD34+ cells are cultured in the presence of GM-CSF and/or IL-4 for a period of time in a range of from 4 days to 18 days (e.g., 5 days to 17 days, 7 days to 16 days, 8 days to 13 days, 9 days to 12 days, 6 days to 15 days, 8 days to 15 days, 10 days to 15 days, 12 days to 15 days, 13 days to 15 days, 5 days to 14 days, 5 days to 12 days, 5 days to 10 days, 5 days to 9 days, 6 days to 8 days, 6 days, 7 days, 8 days, 9 days, 10 days, 12 days, or 14 days). When CD34+ cells are cultured in the presence of GM-CSF and/or IL-4, the GM-CSF can be at a concentration in a range of from 35 ng/ml to 65 ng/ml (35 ng/ml to 65 ng/ml, 40 ng/ml to 60 ng/ml, 45 ng/ml to 50 ng/ml, or 50 ng/ml) and the IL-4 can be at a concentration in a range of from 5 ng/ml to 35 ng/ml (10 ng/ml to 30 ng/ml, 15 ng/ml to 25 ng/ml, 17.5 ng/ml to 22.5 ng/ml, or 20 ng/ml). As an illustrative example, bones can flushed with a saline solution (e.g., phosphate buffered saline (PBS)) and mononuclear cells can be separated from the bone marrow on Ficoll gradients. CD34+ cells can then be isolated/enriched (e.g., using antibody-conjugated magnetic beads) and then cultured in the presence of GM-CSF and IL-4 (as described above). In some cases (e.g., when the cells are mouse cells), DCs can be derived by culturing the cells in GM-CSF. In some cases (e.g., when the cells are human cells), DCs can be derived by culturing the cells in GM-CSF and IL-4.

In some cases, monocytes are used as a source for generating DCs (sometimes referred to as blood derived DCs, blood Mo-DCs, monocyte DCs, and the like). For example, DCs can be generated by culturing adherent cells (monocytes, e.g., bone marrow monocytes, blood monocytes, etc.)(e.g., CD14+ blood monocytes) in the presence of GM-CSF (e.g., at a concentration in a range as described above for BMDC) and/or IL-4 (e.g., at a concentration in a range as described above for BMDC) for a period of time in a range of from 3 days to 9 days (e.g., 4 days to 8 days, 5 days to 7 days, 3 days to 6 days, 4 days to 5 days, 6 days to 8 days, or 7 days). For example, in some cases, mononuclear cells are isolated from blood and enriched for CD11b+ cells (e.g., using magnetic beads). The cells can be sorted for “inflammatory monocytes” (FSC^{lo}/SSC^{lo}/Gr1^{hi}/CD115^{hi}) and/or “patrolling monocytes” (FSC^{lo})/SSC^{lo}/Gr1^{neg}/CD115^{hi}). DCs can then be generated from various types of monocytes by culturing the monocytes in the presence of GM-CSF (e.g., for a period of time in a range of from 3 days to 6 days (e.g., 4 days to 5 days)). In some cases (e.g., when the cells are mouse cells), DCs are derived by culturing the cells in GM-CSF. In some cases (e.g., when the cells are human cells), DCs are derived by culturing the cells in GM-CSF and IL-4. To obtain DC from spleen (a splenic DC), splenocytes can be enriched (e.g., using antibody-coupled magnetic beads) for CD11c⁺ cells and CD11c^{hi}/MHCII^{hi} cells can be sorted/enriched using flow cytometry (e.g., FACS).

In some cases, DC are tumor associated DC (TADC). TADC can be obtained by any convenient method. For example, to obtain DC from tumors (tumor associated DC, TADC), tumors can be digested (e.g., using collagenase and nuclease) and CD11c+ cells can be enriched (e.g., using antibody-conjugated magnetic beads), and Gr1^{neg}/CD11C^{hi}/MHCII^{hi} cells can be sorted/enriched using flow cytometry (e.g., FACS).

Isolated and/or derived DCs (e.g., as described above) can be activated using various factors including, but not limited to TNFα (e.g., 50 ng/ml) and a CD40 ligand (e.g., CD40L) (e.g., 500 ng/ml) (described in further detail below).

For more information regarding dendritic cells and methods of isolating, generating, and/or culturing DC, see: Vassalli, J Transplant. 2013; 2013: 761429: “Dendritic Cell-Based Approaches for Therapeutic Immune Regulation in Solid-Organ Transplantation”; Syme et al., Stem Cells. 2005; 23(1):74-81: “Comparison of CD34 and monocyte-derived dendritic cells from mobilized peripheral blood from cancer patients”; Banchereau et al., Annu Rev Immunol. 2000; 18:767-811: “Immunobiology of dendritic cells”; and U.S. patent application numbers 20130330822; 20130273654; 20130130380; 20120251561; and 20120244620; all of which are hereby incorporated by reference in their entirety.

Macrophages. A macrophage is a type of antigen-presenting cell (APC) of the mammalian immune system. The term “macrophage” as used herein refers to any member of a diverse population of morphologically similar cell types found in lymphoid or non-lymphoid tissues. These cells are characterized by their distinctive morphology and high levels of surface MHC-class II expression. A macrophage is a monocyte-derived phagocyte which is not a dendritic cell or a cell that derives from tissue macrophages by local proliferation. In the body these cells are tissue specific and refer to e. g. Kupffer cells in the liver, alveolar macrophages in the lung, microglia cells in the brain, osteoclasts in the bone etc. The skilled person is aware how to identify macrophage cells, how to isolate macrophage cells from the body of a human or animal, and how to characterize macrophage cells with respect to their subclass and subpopulation (Kruisbeek, 2001; Davies and Gordon 2005 a and b; Zhang et al., 2008; Mosser and Zhang, 2008; Weischenfeldt and Porse, 2008; Ray and Dittel, 2010; Martinez et al., 2008; Jenkins et al., 2011).

Macrophages can be activated by different mechanisms into different subclasses, including, but not limited to M1, M2, M2a, M2b, and M2c subclasses. Whereas the term M1 is used to describe classically activated macrophages that arise due to injury or bacterial infection and IFN-γ activation, M2 is a generic term for numerous forms of macrophages activated differently than M1. The M2 classification has further been divided into subpopulations (Mantovani et al., 2004). The most representative form is M2a macrophages, which commonly occur in helminth infections by exposure to worm induced Th2 cytokines IL-4 and IL-13. M2a macrophages were, among others, shown to be essentially involved in protecting the host from re-infection (Anthony et al., 2006) or in contributing to wound healing and tissue remodeling (Gordon, 2003). Another subpopulation is M2b macrophages that produce high levels of IL-10 and low levels of IL-12 but are not per se anti-inflammatory (Anderson and Mosser, 2002; Edwards et al., 2006). M2b macrophages are elicited by immune complexes that bind to Fc-γ receptors in combination with TLR ligands. Finally, M2c macrophages represent a subtype elicited by IL-10, TGF-β or glucocorticoids (Martinez et al., 2008).

Thus, “M2a macrophages” refers to a macrophage cell that has been exposed to a milieu under Th2 conditions (e g. exposure to Th2 cytokines IL-4 and IL-13) and exhibits a specific phenotype by higher expression of the gene Ym1 and/or the gene CD206 and/or the gene RELM-α and/or the gene Arginase-1. Similarly, “M2b macrophages” refers to a macrophage cell that has been exposed to a milieu of immune complexes in combination with TLR or TNF-alpha stimulation. Said cell is characterized through higher expression of the gene SPHK-1 and/or the gene LIGHT and/or the gene IL-10.

In some cases, the present disclosure refers to a macrophage cell “derived from the body of a patient”. This is meant to designate that either macrophages are obtained from the body of said patient, or macrophage precursor cells are obtained from the body of said patient and subsequently differentiated into macrophage cells in vitro as described in Wahl et al. 2006; Davis and Gordon 2005; Smythies et al., 2006; Zhang et al., 2008; Mosser and Zhang, 2008.

B-Cells.

A B-cell is a type of antigen-presenting cell (APC) of the mammalian immune system. The term “B-cell” as used herein refers to B-cells from any stage of development (e.g., B-stem cells, progenitor B-cells, differentiated B-cells, plasma cells) and from any source including, but not limited to peripheral blood, a region at, in, or near a tumor, lymph nodes, bone marrow, umbilical cord blood, or spleen cells.

B-cell precursors reside in the bone marrow where immature B-cells are produced. B-cell development occurs through several stages, each stage representing a change in the genome content at the antibody loci. In the genomic heavy chain variable region there are three segments, V, D, and J, which recombine randomly, in a process called VDJ rearrangement to produce a unique variable region in the immunoglobulin of each B-cell. Similar rearrangements occur for the light chain variable region except that there are only two segments involved, V and J. After complete rearrangement, the B-cell reaches the IgM+ immature stage in the bone marrow. These immature B-cells present a membrane bound IgM, i.e., BCR, on their surface and migrate to the spleen, where they are called transitional B cells. Some of these cells differentiate into mature B lymphocytes. Mature B-cells expressing the BCR on their surface circulate the blood and lymphatic system performing the role of immune surveillance. They do not produce soluble antibodies until they become fully activated. Each B-cell has a unique receptor protein that will bind to one particular antigen. Once a B-cell encounters its antigen and receives an additional signal from a T helper cell, it can further differentiate into either a plasma B-cell expressing and secreting soluble antibodies or a memory B-cell.

In the context of the present disclosure, the term “B-cell” refers to any B lymphocyte which presents a fully rearranged, i.e., a mature, BCR on its surface. For example, a B-cell in the context of the present invention may be an immature or a mature B-cell. In some cases, the B-cell is a naïve B-cell, i.e., a B-cell that has not been exposed to the antigen specifically recognized by the BCR on the surface of said B-cell. In some embodiments, the B-cells are CD19+ B-cells, i.e., express CD19 on their surface. In some cases, the B-cells in the context of the present invention are CD19+ B-cells and express a fully rearranged BCR on their surface. The B-cells may also be CD20+ or CD21+ B-cells. In some cases, the CD20+ or CD21+ B-cells carry a BCR on their surface. In some embodiments, the B-cells are memory B-cells, such as IgG+ memory B cells.

### Treatment

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those with cancer, e.g. those having tumors) as well as those in which prevention is desired (e.g., those with increased susceptibility to cancer; those with pre-cancerous tumors, lesions; those suspected of having cancer; etc.).

The terms “recipient”, “individual”, “subject”, “host”, and “patient”, are used interchangeably herein and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

A therapeutic treatment is one in which the subject is inflicted prior to administration and a prophylactic treatment is one in which the subject is not inflicted prior to administration. In some embodiments, the subject has an increased likelihood of becoming inflicted or is suspected of having an increased likelihood of becoming inflicted (e.g., relative to a standard, e.g., relative to the average individual, e.g., a subject may have a genetic predisposition to cancer and/or a family history indicating increased risk of cancer), in which case the treatment can be a prophylactic treatment. In some cases, the term “vaccination” is used to describe a prophylactic treatment. For example, in some cases where the subject being treated has not been diagnosed as having cancer (e.g., the subject has an increased likelihood of becoming inflicted, is suspected of having an increased likelihood of becoming inflicted)(e.g., a subject may have a genetic predisposition to cancer and/or a family history indicating increased risk of cancer), the subject can be vaccinated (treated such that the treatment is a prophylactic treatment) by performing one or more of the subject methods.

In some cases where the subject being treated has not been diagnosed as having cancer (e.g., the subject has an increased likelihood of becoming inflicted, is suspected of having an increased likelihood of becoming inflicted)(e.g., a subject may have a genetic predisposition to cancer and/or a family history indicating increased risk of cancer), the subject can be vaccinated (treated such that the treatment is a prophylactic treatment) by performing one or more of the subject methods.

### Individuals to be Treated and ‘Cancer Cells’

In some embodiments, the individual to be treated is an individual with cancer. As used herein “cancer” includes any form of cancer (e.g., leukemia; acute myeloid leukemia (AML); acute lymphoblastic leukemia (ALL); lymphomas; mesothelioma (MSTO); minimal residual disease; solid tumor cancers, e.g., lung, prostate, breast, bladder, colon, ovarian, pancreas, kidney, glioblastoma, medulloblastoma, leiomyosarcoma, and head & neck squamous cell carcinomas, melanomas; etc.), including both primary and metastatic tumors; and the like. In some cases, the individual has recently undergone treatment for cancer (e.g., radiation therapy, chemotherapy, surgical resection, etc.) and are therefore at risk for recurrence. Any and all cancers are suitable cancers to be treated by the subject methods, compositions, and kits.

The terms “cancer,” “neoplasm,” and “tumor” are used herein to refer to cells which exhibit autonomous, unregulated growth, such that they exhibit an aberrant growth phenotype characterized by a significant loss of control over cell proliferation. Cells of interest for detection, analysis, and/or treatment in the present disclosure include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell (e.g., from any of the cancers for which an individual can be treated, e.g., isolated from an individual having cancer) or is derived from a cancer cell e.g. clone of a cancer cell. For example, a cancer cell can be from an established cancer cell line, can be a primary cell isolated from an individual with cancer, can be a progeny cell from a primary cell isolated from an individual with cancer, and the like. In some cases, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, myelomas, etc., and circulating cancers such as leukemias.

As used herein “cancer” includes any form of cancer, including but not limited to solid tumor cancers (e.g., lung, prostate, breast, bladder, colon, ovarian, pancreas, kidney, liver, glioblastoma, medulloblastoma, leiomyosarcoma, head & neck squamous cell carcinomas, melanomas, neuroendocrine; etc.) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors. Any cancer is a suitable cancer to be treated by the subject methods and compositions.

Carcinomas are malignancies that originate in the epithelial tissues. Epithelial cells cover the external surface of the body, line the internal cavities, and form the lining of glandular tissues. Examples of carcinomas include, but are not limited to: adenocarcinoma (cancer that begins in glandular (secretory) cells), e.g., cancers of the breast, pancreas, lung, prostate, and colon can be adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, skin, etc.

Soft tissue tumors are a highly diverse group of rare tumors that are derived from connective tissue. Examples of soft tissue tumors include, but are not limited to: alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; and pleomorphic adenoma of soft tissue; and neoplasias derived from fibroblasts, myofibroblasts, histiocytes, vascular cells/endothelial cells and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to: askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; undifferentiated pleomorphic sarcoma, and the like).

A teratoma is a type of germ cell tumor that may contain several different types of tissue (e.g., can include tissues derived from any and/or all of the three germ layers: endoderm, mesoderm, and ectoderm), including for example, hair, muscle, and bone. Teratomas occur most often in the ovaries in women, the testicles in men, and the tailbone in children.

Melanoma is a form of cancer that begins in melanocytes (cells that make the pigment melanin). It may begin in a mole (skin melanoma), but can also begin in other pigmented tissues, such as in the eye or in the intestines.

Leukemias are cancers that start in blood-forming tissue, such as the bone marrow, and causes large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to: Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One kind is Hodgkin lymphoma (HL), which is marked by the presence of a type of cell called the Reed-Sternberg cell. There are currently 6 recognized types of HL. Examples of Hodgkin lymphomas include: nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to: AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to: gliomas (e.g., glioblastomas, astrocytomas, oligodendrogliomas, ependymomas, and the like), meningiomas, pituitary adenomas, vestibular schwannomas, primitive neuroectodermal tumors (medulloblastomas), etc.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

### Co-Administration

In some cases, a subject Dectin-2 stimulating agent (e.g., a direct Dectin-2 stimulating agent such as a composition that includes a Dectin-2 binding glycopolymer such as a glycopolypeptide, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) (e.g., formulated as a pharmaceutical composition) is co-administered with another agent such as a cancer therapeutic drug (e.g., a tumor-directed antibody). Such administration may involve concurrent (i.e. at the same time), prior, or subsequent administration of the drug/antibody with respect to the administration of an agent or agents of this disclosure. A person of ordinary skill in the art would have no difficulty determining the appropriate timing, sequence and dosages of administration for particular drugs and compositions of the present disclosure. In some cases, a Dectin-2 stimulating agent (e.g., a direct Dectin-2 stimulating agent such as a composition that includes a Dectin-2 binding glycopolymer such as a glycopolypeptide, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) is formulated with one or more agents that potentiate activity, or that otherwise increase the therapeutic effect (such as an immunomodulatory agent, a tumor-directed antibody, and the like).

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

Treatment with a subject Dectin-2 stimulating agent (e.g., a direct Dectin-2 stimulating agent such as a composition that includes a Dectin-2 binding glycopolymer such as a glycopolypeptide, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) can be combined with chemotherapy, radiotherapy, and/or other immunotherapies to enhance effect.

In some cases two or more subject Dectin-2 stimulating agents are co-administered with one another. For example, a non-plant derived naturally existing ligand for Dectin-2 can be co-administered with one or more of: (i) a synthetic Dectin-2 stimulating glycopolymer or mimetic thereof (e.g., a glycopolypeptide); (ii) a Dectin-2 stimulating anti-Dectin-2 antibody, and (iii) an alpha-mannosidase class 1 inhibitor. In some cases, a synthetic Dectin-2 stimulating glycopolymer or mimetic thereof (e.g., a glycopolypeptide) can be co-administered with one or more of: (i) a non-plant derived naturally existing ligand for Dectin-2; (ii) a Dectin-2 stimulating anti-Dectin-2 antibody, and (iii) an alpha-mannosidase class 1 inhibitor. In some cases, a Dectin-2 stimulating anti-Dectin-2 antibody can be co-administered with one or more of: (i) a non-plant derived naturally existing ligand for Dectin-2; (ii) a synthetic Dectin-2 stimulating glycopolymer or mimetic thereof (e.g., a glycopolypeptide), and (iii) an alpha-mannosidase class 1 inhibitor. In some cases, an alpha-mannosidase class 1 inhibitor can be co-administered with one or more of: (i) a non-plant derived naturally existing ligand for Dectin-2; (ii) a synthetic Dectin-2 stimulating glycopolymer or mimetic thereof (e.g., a glycopolypeptide), and (iii) a Dectin-2 stimulating anti-Dectin-2 antibody.

One class of cytotoxic agents that can be used in combination with (co-administered with) a Dectin-2 stimulating agent are chemotherapeutic agents. Exemplary chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, duocarmycin, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, saproin, tamoxifen, taxol, topotecan hydrochloride, trastuzumab, vinblastine, vincristine and vinorelbine tartrate.

In some cases, a subject Dectin-2 stimulating agent (e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) is used in a combination therapy (is co-administered) with a cancer targeting agent (e.g., an agent that specifically binds a cancer antigen, e.g., a cell-specific antibody selective for a tumor cell marker). Any convenient cancer cell targeting agent can be used. In some cases, the cancer cell targeting agent is a specific binding agent (e.g., a polypeptide such as an antibody that includes an antigen binding region specific for a cancer antigen) that specifically binds a cancer antigen of cancer cells (e.g., CD19, CD20, CD22, CD24, CD25, CD30, CD33, CD38, CD44, CD47, CD52, CD56, CD70, CD96, CD97, CD99, CD123, CD279 (PD-1), CD274 (PD-L1), EpCam, EGFR, 17-1A, HER2, CD117, C-Met, PTHR2, HAVCR2 (TIM3), and SIRPA). As such, in some cases, a subject method includes co-administering a subject Dectin-2 stimulating agent (e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) and a cancer cell targeting agent that is a specific binding agent (e.g., a polypeptide such as an antibody that includes an antigen binding region specific for a cancer antigen) that specifically binds an antigen (e.g., a cancer antigen) selected from: CD19, CD20, CD22, CD24, CD25, CD30, CD33, CD38, CD44, CD47, CD52, CD56, CD70, CD96, CD97, CD99, CD123, CD279 (PD-1), CD274 (PD-L1), EpCam, EGFR, 17-1A, HER2, CD117, C-Met, PTHR2, HAVCR2 (TIM3), and SIRPA.

In some cases, a subject Dectin-2 stimulating agent (e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) is used in a combination therapy (is co-administered) with one or more of: cetuximab (binds EGFR), panitumumab (binds EGFR), rituximab (binds CD20), trastuzumab (binds HER2), pertuzumab (binds HER2), alemtuzumab (binds CD52), brentuximab (binds CD30), tositumomab, ibritumomab, gemtuzumab, ibritumomab, and edrecolomab (binds 17-1A).

In some cases, a subject Dectin-2 stimulating agent (e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor), is used in a combination therapy (is co-administered) with an immunomodulatory agent. Any convenient immunomodulatory agent can be used. In some cases, the immunomodulatory agent is selected from: an anti-CTLA4 antibody; an anti-PD-1/PD-L1 agent (e.g., an anti-PD-1 antibody, a PD-1-binding reagent such as a PD-L1 or PD-L2 ectodomain, an anti-PD-L1 antibody, a PD-L1-binding reagent such as a PD-1 ectodomain, and the like); a CD40 agonist (e.g., CD40L); a 4-1BB modulator (e.g., a 4-1BB-agonist); an anti-CD47/SIRPA agent (e.g., an anti-CD47 antibody, a CD47-binding reagent such as a SIRPA ectodomain, an anti-SIRPA antibody, a SIRPA-binding reagent such as a CD47 ectodomain, and the like); an inhibitor of TIM3 and/or CEACAM1; an inhibitor of TIM3 and/or CEACAM1; an inhibitor of BTLA and/or CD160; and the like.

Suitable agents that can be co-administered with a subject Dectin-2 stimulating agent (e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) include but are not limited to (i) a CD40 agonist (e.g., CD40L and/or an agonistic anti-CD40 antibody), (ii) a proinflammatory cytokine (e.g., TNFα, IL-1α, IL-1β, IL-19, interferon gamma (IFNγ), and the like), (iii) a Toll-like receptor (TLR) agonist (e.g., a CpG ODN, polyinosinic:polycytidylic acid (“poly I:C”, a TLR-3 agonist), etc.), (iv) an indoleamine 2,3-dioxygenase (IDO) inhibitor, (v) an agent that neutralizes checkpoint molecules (i.e., a checkpoint blockade agent) (e.g., an anti-CTLA-4 antibody, e.g., Ipilimumab; an anti-PD-1 antibody; an anti-PD-L1 antibody, and the like), (vi) a T cell-related co-stimulatory molecule (e.g., CD27, CD28, 4-BBL, and the like), (vii) an NFkB activator, and (viii) an agent that induces Dectin-2 expression by myeloid cells (e.g., TNFα, IFNγ, granulocyte macrophage colony-stimulating factor (GM-CSF), and the like).

Thus, in some cases, one of the co-administered therapeutic agents is a composition that includes a subject Dectin-2 stimulating agent (e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor), and it is co-administered with one or more agents selected from: (i) a CD40 agonist (e.g., CD40L and/or an agonistic anti-CD40 antibody), (ii) a proinflammatory cytokine (e.g., TNFα, IL-1α, IL-1β, IL-19, interferon gamma (IFNγ), and the like), (iii) a Toll-like receptor (TLR) agonist (e.g., a CpG ODN, polyinosinic:polycytidylic acid (“poly I:C”, a TLR-3 agonist), etc.), (iv) an indoleamine 2,3-dioxygenase (IDO) inhibitor, (v) an agent that neutralizes checkpoint molecules (e.g., an anti-CTLA-4 antibody, e.g., Ipilimumab; an anti-PD-1 antibody; an anti-PD-L1 antibody), (vi) a T cell-related co-stimulatory molecule (e.g., CD27, CD28, 4-BBL, and the like), (vii) an NFkB activator, and (viii) an agent that induces Dectin-2 expression by myeloid cells (e.g., TNFα, IFNγ, granulocyte macrophage colony-stimulating factor (GM-CSF), and the like). In some cases, the proinflammatory cytokine is IL-I, IL-2, IL-3, IL-4, IL-6, IL-7, IL-9, IL-10, IL-12, IL-15, IL-18, IL-21, TNFα, IL-1α, IL-1β, IL-19, IFN-α, IFN-β, IFN-γ, G-CSF, or GM-CSF. In some cases, one of the co-administered therapeutic agents is a composition that includes a subject Dectin-2 stimulating agent (e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor), and it is co-administered with GM-CSF, TNFα, or IFNγ. In some cases, one of the co-administered therapeutic agents is a composition that includes a subject Dectin-2 stimulating agent (e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor), and it is co-administered with GM-CSF. In some cases, one of the co-administered therapeutic agents is a composition that includes a subject Dectin-2 stimulating agent (e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor), and it is co-administered with IFNγ.

In some cases, the subject therapeutic agent (e.g., a composition that includes a subject Dectin-2 stimulating agent, e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) is co-administered with a CD40 agonist (e.g., CD40L and/or an agonistic anti-CD40 antibody). In some cases, the subject therapeutic agent (e.g., a composition that includes a subject Dectin-2 stimulating agent, e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) is co-administered with a proinflammatory cytokine (e.g., TNFα, IL-1α, IL-1β, IL-19, interferon gamma (IFNγ), and the like).

In some cases, the subject therapeutic agent (e.g., a composition that includes a subject Dectin-2 stimulating agent, e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) is co-administered with a Toll-like receptor (TLR) agonist (e.g., a CpG ODN, polyinosinic:polycytidylic acid (“poly I:C”, a TLR-3 agonist), etc.). In some cases, the subject therapeutic agent (e.g., a composition that includes a subject Dectin-2 stimulating agent, e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) is co-administered with an indoleamine 2,3-dioxygenase (IDO) inhibitor. In some cases, the subject therapeutic agent (e.g., a composition that includes a subject Dectin-2 stimulating agent, e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) is co-administered with an agent that neutralizes checkpoint molecules (e.g., an anti-CTLA-4 antibody, e.g., Ipilimumab; an anti-PD-1 antibody; an anti-PD-L1 antibody).

In some cases, the subject therapeutic agent (e.g., a composition that includes a subject Dectin-2 stimulating agent, e.g., a direct Dectin-2 stimulating agent such as a composition that includes a Dectin-2 binding glycopolymer, e.g., oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) is co-administered with a T cell-related co-stimulatory molecule (e.g., CD27, CD28, 4-BBL, and the like). In some cases, the subject therapeutic agent (e.g., a composition that includes a subject Dectin-2 stimulating agent, e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) is co-administered with an NFkB activator. In some cases, the subject therapeutic agent (e.g., a composition that includes a subject Dectin-2 stimulating agent, e.g., a direct Dectin-2 stimulating agent such as a composition that includes a Dectin-2 binding glycopolymer, e.g., an oligomannose glycopolypeptide, a Dectin-2 binding glycan, e.g. mannan polysaccharide or another oligomannose glycan, and/or a Dectin-2 antibody, or an indirect Dectin-2 stimulating agent such as an alpha-mannosidase class I inhibitor) is co-administered with an agent that induces Dectin-2 expression by myeloid cells (e.g., TNFα, IFNγ, granulocyte macrophage colony-stimulating factor (GM-CSF), and the like).

Treatment with a Dectin-2 stimulating agent may be combined (co-administered) with other active agents, such as antibiotics, cytokines, anti-viral agents, etc. Classes of antibiotics include penicillins, e.g. penicillin G, penicillin V, methicillin, oxacillin, carbenicillin, nafcillin, ampicillin, etc.; penicillins in combination with β-lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. Cytokines may also be included, e.g. interferon γ, tumor necrosis factor α, interleukin 12, etc. Antiviral agents, e.g. acyclovir, gancyclovir, etc., may also be used in treatment.

Administering Cells and/or Compositions.

In some cases, cells (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) are transplanted into an individual (i.e., administered to the individual). In some cases, the cells are cultured for a period of time prior to. Cells (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) can be provided to the individual (i.e., administered into the individual) alone or with a suitable substrate or matrix, e.g. to support their growth and/or organization in the tissue to which they are being transplanted (e.g., target organ, tumor tissue, blood stream, and the like). In some embodiments, the matrix is a scaffold (e.g., an organ scaffold). In some embodiments, 1×10³ or more cells will be administered, for example 5×10³ or more cells, 1×10⁴ or more cells, 5×10⁴ or more cells, 1×10⁵ or more cells, 5×10⁵ or more cells, 1×10⁶ or more cells, 5×10⁶ or more cells, 1×10⁷ or more cells, 5×10⁷ or more cells, 1×10⁸ or more cells, 5×10⁸ or more cells, 1×10⁹ or more cells, 5×10⁹ or more cells, or 1×10¹⁰ or more cells. In some embodiments, subject cells are administered into the individual on microcarriers (e.g., cells grown on biodegradable microcarriers).

Subject cells (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) and/or compositions (e.g., a Dectin-2 stimulating composition that includes a subject Dectin-2 stimulating agent) can be administered in any physiologically acceptable excipient (e.g., William's E medium), e.g., where transplanted cells may find an appropriate site for survival and function (e.g., organ reconstitution). The cells and/or compositions (e.g., a Dectin-2 stimulating composition that includes a subject Dectin-2 stimulating agent) may be introduced by any convenient method (e.g., injection, catheter, or the like). The cells and/or compositions can be encapsulated into liposomes or other biodegradable constructs. In some cases, a subject Dectin-2 stimulating agent is administered in or conjugated to a liposome, a microparticle, or a nanoparticle.

The subject cells (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) and/or compositions (e.g., a Dectin-2 stimulating composition that includes a subject Dectin-2 stimulating agent) can be introduced to an individual (i.e., administered to the individual) via any of the following routes: parenteral, subcutaneous (s.c.), intravenous (i.v.), intracranial (i.c.), intraspinal, intraocular, intradermal (i.d.), intramuscular (i.m.), intralymphatic (i.l.), or into spinal fluid. The cells and/or compositions can be introduced to an individual systemically (e.g., parenteral, s.c., i.v., orally, and the like) or locally (e.g., direct local injection, local injection into or near a tumor and/or a site of tumor resection, and the like). The cells and/or compositions can be introduced by injection (e.g., systemic injection, direct local injection, local injection into or near a tumor and/or a site of tumor resection, etc.), catheter, or the like. Examples of methods for local delivery (e.g., delivery to a tumor, cancer site, and/or a site of tumor resection) include, e.g., by bolus injection, e.g. by a syringe, e.g. into a joint, tumor, or organ, or near a joint, tumor, or organ; e.g., by continuous infusion, e.g. by cannulation, e.g. with convection (see e.g. US Application No. 20070254842, incorporated here by reference); or by implanting a device upon which cells have been reversibly affixed (see e.g. US Application Nos. 20080081064 and 20090196903, incorporated herein by reference).

The subject cells (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) and/or compositions (e.g., a Dectin-2 stimulating composition that includes a subject Dectin-2 stimulating agent) can be introduced to an individual by any suitable means, including topical, oral, parenteral, intrapulmonary, and intranasal, and the like. Parenteral infusions include intramuscular, intravenous (bolus or slow drip), intraarterial, intraperitoneal, intrathecal or subcutaneous administration. For example, the subject cells and compositions can be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the disclosure, by such means as depot injections or erodible implants. Localized delivery is also contemplated, e.g., delivery via a catheter to one or more arteries, such as the renal artery or a vessel supplying a localized tumor.

In some cases a subject cell (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) and/or composition (e.g., a Dectin-2 stimulating composition that includes a subject Dectin-2 stimulating agent) is administered by local injection into or near a tumor and/or a site of tumor resection. In some cases, a subject cell (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) and/or composition (e.g., a Dectin-2 stimulating composition that includes a subject Dectin-2 stimulating agent) is administered by local injection into or near a tumor and/or a site of tumor resection (e.g., in some cases in a liposome, a microparticle, or a nanoparticle).

The number of administrations of treatment to a subject may vary. Introducing cells and/or compositions into an individual (administering cells and/or compositions) may be a one-time event; but in certain situations, such treatment may elicit improvement for a limited period of time and require an on-going series of repeated treatments. In other situations, multiple administrations of cells and/or compositions may be required before an effect is observed. As will be readily understood by one of ordinary skill in the art, the exact protocols depend upon the disease or condition, the stage of the disease and parameters of the individual being treated.

A “therapeutically effective dose” or “therapeutic dose” is an amount sufficient to effect desired clinical results (i.e., achieve therapeutic efficacy). A therapeutically effective dose can be administered in one or more administrations. For purposes of this disclosure, a therapeutically effective dose of subject cells (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) and/or compositions (e.g., a Dectin-2 stimulating composition that includes a subject Dectin-2 stimulating agent) is an amount that is sufficient, when administered to (e.g., transplanted into) the individual, to palliate, ameliorate, stabilize, reverse, prevent, slow or delay the progression of the disease state (e.g., reduce: the number of cancer cells, tumor size, tumor growth, tumor presence, cancer presence, etc.) by, for example, inducing an immune response against antigenic cells (e.g., cancer cells).

A therapeutically effective dose of a Dectin-2 stimulating composition can depend on the specific agent used, but is usually 8 mg/kg body weight or more (e.g., 8 mg/kg or more, 10 mg/kg or more, 15 mg/kg or more, 20 mg/kg or more, 25 mg/kg or more, 30 mg/kg or more, 35 mg/kg or more, or 40 mg/kg or more) for each agent, or from 10 mg/kg to 40 mg/kg (e.g., from 10 mg/kg to 35 mg/kg, or from 10 mg/kg to 30 mg/kg) for each agent. The dose required to achieve and/or maintain a particular serum level is proportional to the amount of time between doses and inversely proportional to the number of doses administered. Thus, as the frequency of dosing increases, the required dose decreases. The optimization of dosing strategies will be readily understood and practiced by one of ordinary skill in the art. For all therapeutically effective doses listed above, when more than one agent is used (e.g., two or Dectin-2 stimulating agents, a Dectin-2 stimulating agent co-administered with another anti-cancer agent such as a tumor targeting antibody or an immunomodulatory agent), the dose for each agent can be independent from the other agent. As an illustrative example (to illustrate the independence of the doses), in one case, a therapeutic dose of a subject Dectin-2 stimulating agent might be from 75 ug/ml to 250 ug/ml while a therapeutic dose of an immunomodulatory agent might be from 40 ug/ml to 100 ug/ml.

Dosage and frequency may vary depending on the half-life of the Dectin-2 stimulating agent in the patient. It will be understood by one of skill in the art that such guidelines will be adjusted for the molecular weight of the active agent, e.g. in the use of antibody fragments, in the use of Dectin-2 stimulating agents. The dosage may also be varied for localized administration, e.g. intranasal, inhalation, etc., or for systemic administration, e.g. i.m., i.p., i.v., and the like.

In some embodiments, a therapeutically effective dose of cells (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) is 1×10³ or more cells (e.g., 5×10³ or more, 1×10⁴ cells, 5×10⁴ or more, 1×10⁵ or more, 5×10⁵ or more, 1×10⁶ or more, 2×10⁶ or more, 5×10⁶ or more, 1×10⁷ cells, 5×10⁷ or more, 1×10⁸ or more, 5×10⁸ or more, 1×10⁹ or more, 5×10⁹ or more, or 1×10¹⁰ or more).

In some embodiments, a therapeutically effective dose of cells (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) is in a range of from 1×10³ cells to 1×10¹⁰ cells (e.g., from 5×10³ cells to 1×10¹⁰ cells, from 1×10⁴ cells to 1×10¹⁰ cells, from 5×10⁴ cells to 1×10¹⁰ cells, from 1×10⁵ cells to 1×10¹⁰ cells, from 5×10⁵ cells to 1×10¹⁰ cells, from 1×10⁶ cells to 1×10¹⁰ cells, from 5×10⁶ cells to 1×10¹⁰ cells, from 1×10⁷ cells to 1×10¹⁰ cells, from 5×10⁷ cells to 1×10¹⁰ cells, from 1×10⁸ cells to 1×10¹⁰ cells, from 5×10⁸ cells to 1×10¹⁰, from 5×10³ cells to 5×10⁹ cells, from 1×10⁴ cells to 5×10⁹ cells, from 5×10⁴ cells to 5×10⁹ cells, from 1×10⁵ cells to 5×10⁹ cells, from 5×10⁵ cells to 5×10⁹ cells, from 1×10⁶ cells to 5×10⁹ cells, from 5×10⁶ cells to 5×10⁹ cells, from 1×10⁷ cells to 5×10⁹ cells, from 5×10⁷ cells to 5×10⁹ cells, from 1×10⁸ cells to 5×10⁹ cells, from 5×10⁸ cells to 5×10⁹, from 5×10³ cells to 1×10⁹ cells, from 1×10⁴ cells to 1×10⁹ cells, from 5×10⁴ cells to 1×10⁹ cells, from 1×10⁵ cells to 1×10⁹ cells, from 5×10⁵ cells to 1×10⁹ cells, from 1×10⁶ cells to 1×10⁹ cells, from 5×10⁶ cells to 1×10⁹ cells, from 1×10⁷ cells to 1×10⁹ cells, from 5×10⁷ cells to 1×10⁹ cells, from 1×10⁸ cells to 1×10⁹ cells, from 5×10⁸ cells to 1×10⁹, from 5×10³ cells to 5×10⁸ cells, from 1×10⁴ cells to 5×10⁸ cells, from 5×10⁴ cells to 5×10⁸ cells, from 1×10⁵ cells to 5×10⁸ cells, from 5×10⁵ cells to 5×10⁸ cells, from 1×10⁶ cells to 5×10⁸ cells, from 5×10⁶ cells to 5×10⁸ cells, from 1×10⁷ cells to 5×10⁸ cells, from 5×10⁷ cells to 5×10⁸ cells, or from 1×10⁸ cells to 5×10⁸ cells).

In some embodiments, the concentration of cells (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) to be administered is in a range of from 1×10⁵ cells/ml to 1×10⁹ cells/ml (e.g., from 1×10⁵ cells/ml to 1×10⁸ cells/ml, from 5×10⁵ cells/ml to 1×10⁸ cells/ml, from 5×10⁵ cells/ml to 5×10⁷ cells/ml, from 1×10⁶ cells/ml to 1×10⁸ cells/ml, from 1×10⁶ cells/ml to 5×10⁷ cells/ml, from 1×10⁶ cells/ml to 1×10⁷ cells/ml, from 1×10⁶ cells/ml to 6×10⁶ cells/ml, or from 2×10⁶ cells/ml to 8×10⁶ cells/ml).

In some embodiments, the concentration of cells (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) to be administered is 1×10⁵ cells/ml or more (e.g., 1×10⁵ cells/ml or more, 2×10⁵ cells/ml or more, 3×10⁵ cells/ml or more, 4×10⁵ cells/ml or more, 5×10⁵ cells/ml or more, 6×10⁵ cells/ml or more, 7×10⁵ cells/ml or more, 8×10⁵ cells/ml or more, 9×10⁵ cells/ml or more, 1×10⁶ cells/ml or more, 2×10⁶ cells/ml or more, 3×10⁶ cells/ml or more, 4×10⁶ cells/ml or more, 5×10⁶ cells/ml or more, 6×10⁶ cells/ml or more, 7×10⁶ cells/ml or more, or 8×10⁶ cells/ml or more).

The cells (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) and/or compositions (e.g., a Dectin-2 stimulating composition that includes a subject Dectin-2 stimulating agent) of this disclosure (i.e., subject cells and/or subject compositions) can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types.

As noted above, a Dectin-2 stimulating agent can be formulated with a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

A Dectin-2 stimulating agent can be administered as a pharmaceutical composition comprising an active therapeutic agent and another pharmaceutically acceptable excipient. The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. A carrier may also bear a Dectin-2 stimulating agent by non-covalent associations, such as non-covalent bonding or by encapsulation. The nature of the carrier can be either soluble or insoluble for purposes of the invention. Those skilled in the art will know of other suitable carriers for binding Dectin-2 stimulating agents, or will be able to ascertain such, using routine experimentation.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

A subject Dectin-2 stimulating agent can be delivered (administered) using a convenient delivery method. For example, to improve the biodistribution of cancer drugs, nanoparticles have been designed for optimal size and surface characteristics to increase their circulation time in the bloodstream. They are also able to carry their loaded active drugs to cancer cells by selectively using the unique pathophysiology of tumors, such as their enhanced permeability and retention effect and the tumor microenvironment. In addition to this passive targeting mechanism, active targeting strategies using ligands or antibodies directed against selected tumor targets amplify the specificity of these therapeutic nanoparticles. (see, e.g., Cho et al., Clin Cancer Res. 2015 Oct. 15; 21(20):4499-501). In some cases, a subject Dectin-2 stimulating agent is administered to an individual using a noncarrier. Examples of nanocarriers for delivery of a subject Dectin-2 stimulating agent include but are not limited to: (a) polymeric nanoparticles in which drugs are conjugated to or encapsulated in polymers; (b) polymeric micelles: amphiphilic block copolymers that form to nanosized core/shell structure in aqueous solution (the hydrophobic core region serves as a reservoir for hydrophobic drugs, whereas hydrophilic shell region stabilizes the hydrophobic core and renders the polymer to be water-soluble); (c) dendrimers: synthetic polymeric macromolecule of nanometer dimensions, which is composed of multiple highly branched monomers that emerge radially from the central core; (d) liposomes: self-assembling structures composed of lipid bilayers in which an aqueous volume is entirely enclosed by a membranous lipid bilayer; (e) viral-based nanoparticles: in general structure are the protein cages, which are multivalent, self-assembles structures; and (f) carbon nanotubes: carbon cylinders composed of benzene rings.

Toxicity of the Dectin-2 stimulating agents can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

Cells of the subject methods and compositions (e.g., myeloid cells in which Dectin-2 has been stimulated; APCs in which Dectin-2 has been stimulated; loaded APCs, e.g., loaded DCs; loaded macrophages; loaded B-cells; and/or contacted/stimulated T cells) may be genetically modified to enhance survival, control proliferation, and the like. Cells may be genetically altered by transfection or transduction with a suitable vector, homologous recombination, or other appropriate technique, so that they express a gene of interest. In some embodiments, a selectable marker is introduced, to provide for greater purity of the desired cell.

For further elaboration of general techniques useful in the practice of this disclosure, the practitioner can refer to standard textbooks and reviews in cell biology, tissue culture, and embryology. With respect to tissue culture and stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

### Kits

Also provided are kits for use in the subject methods. The subject kits include any combination of components and compositions for performing the subject methods. In some embodiments, a kit can include one or more of the following: a subject Dectin-2 stimulating agent (e.g., a non-plant derived naturally existing ligand for Dectin-2 such as mannan polysaccharide or another oligomannose glycan; a non-plant derived naturally existing ligand for Dectin-2 such as a fungal cell wall extract; a synthetic Dectin-2 stimulating glycopolymer (e.g., a glycopolypeptide); a Dectin-2 stimulating anti-Dectin-2 antibody such as a soluble antibody (e.g., monoclonal antibody) or an antibody that is immobilized on a solid support; an alpha-mannosidase class 1 inhibitor such as kifunensine; or 1-deoxymannojirimycin, or an RNAi agent or gene editing agent that specifically reduces expression of one or more proteins selected from: MAN1B1, MAN1A1, MAN1A2, and MAN1C1; etc.); components for the isolation, culture, survival, or administration of APC, e.g., DC, and/or T cells; reagents (e.g., buffers) for contacting an APC, e.g., DC; reagents (e.g., buffers) for contacting a T cell; reagents (e.g., buffers) for contacting a target antigen with a subject antibody composition to produce an immune complex; and any combination thereof.

In some embodiments, the kit comprises a direct Dectin-2 stimulating agent (e.g., a naturally existing ligand for Dectin-2; a non-plant derived naturally existing ligand for Dectin-2 such as mannan polysaccharide or another oligomannose glycan; a non-plant derived naturally existing ligand for Dectin-2 such as a fungal cell wall extract; a synthetic Dectin-2 stimulating glycopolymer (e.g., a glycopolypeptide); a Dectin-2 stimulating anti-Dectin-2 antibody such as a soluble antibody (e.g., monoclonal antibody) or an antibody that is immobilized on a solid support) and a pharmaceutical excipient. In some embodiments, the kit comprises an indirect Dectin-2 stimulating agent (e.g., an alpha-mannosidase class 1 inhibitor such as kifunensine; or 1-deoxymannojirimycin, or an RNAi agent or gene editing agent that specifically reduces expression of one or more proteins selected from: MAN1B1, MAN1A1, MAN1A2, and MAN1C1) and a pharmaceutical excipient.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-45 are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of treating an individual with cancer, the method comprising administering to the individual a Dectin-2 stimulating composition comprising one or more Dectin-2 stimulating agents selected from:

(a) a non-plant derived naturally existing ligand for Dectin-2;

(b) a synthetic Dectin-2 stimulating glycopolymer;

(c) a Dectin-2 stimulating anti-Dectin-2 antibody, and

(d) an alpha-mannosidase class 1 inhibitor,

wherein Dectin-2 signaling is stimulated in myeloid cells thereby stimulating an anti-cancer immune response in the individual.
2. The method according to 1, wherein the Dectin-2 stimulating composition comprises a fungal cell wall extract comprising one or more naturally existing glycoproteins that stimulate Dectin-2 signaling.
3. The method according to 1, wherein the Dectin-2 stimulating composition comprises a non-plant derived naturally existing Dectin-2 ligand that is a glycoprotein isolated from a fungal cell wall extract.
4. The method according to 2 or 3, wherein the cell wall extract is an M. furfur cell wall extract.
5. The method according to 1, wherein the Dectin-2 stimulating composition comprises an extract from one or more of: Candida albicans, Schistosoma mansoni, Mycobacterium tuberculosis, and Dermatophagoides farina, wherein the extract comprises one or more naturally existing glycoproteins that stimulate Dectin-2 signaling.
6. The method according to 1, wherein the non-plant derived naturally existing ligand for Dectin-2 comprises mannan polysaccharide, an oligomannose glycan, and/or a fungal cell wall extract.
7. The method according to any of 1-6, wherein the Dectin-2 stimulating composition comprises an anti-Dectin-2 antibody.
8. The method according to 7, wherein the anti-Dectin-2 antibody is immobilized on a solid support.
9. The method according to 8, wherein the solid support is a nanoparticle or microparticle.
10. The method according to any of 1-9, wherein the Dectin-2 stimulating composition comprises kifunensine, which is an alpha-mannosidase class 1 inhibitor.
11. The method according to any of 1-10, wherein said administration comprises local administration.
12. The method according to any of 1-10, wherein said administration comprises systemic administration.
13. The method according to any of 1-12, wherein said administration includes co-administration with one or more of: a CD40 agonist, GM-CSF, TNFα, and IFNγ.
14. A method of treating an individual with cancer, the method comprising:
contacting a cancer cell from the individual with an alpha-mannosidase class 1 inhibitor in vitro or ex vivo, and introducing the contacted cancer cell into the individual.
15. The method according to 14, wherein the contacted cancer cell is administered locally into a tumor of the individual.
16. A method of stimulating an antigen presenting cell (APC), the method comprising:

(a) contacting in vitro or ex vivo a cancer cell with an alpha-mannosidase class 1 inhibitor to produce an inhibitor-contacted cancer cell; and

(b) contacting an APC with the inhibitor-contacted cancer cell.

17. The method according to 16, wherein the alpha-mannosidase class 1 inhibitor is an RNAi agent or gene editing agent that specifically reduces expression of one or more proteins selected from: MAN1B1, MAN1A1, MAN1A2, and MAN1C1.
18. The method according to 16, wherein the alpha-mannosidase class 1 inhibitor is selected from: kifunensine and 1-deoxymannojirimycin.
19. The method according to any of 16-18, wherein the APC is a dendritic cell or a macrophage.
20. The method according to any of 16-19 comprising, after step (b), introducing the contacted APC into the individual.
21. The method according to any of 16-20 comprising, after step (b), contacting a T cell with the contacted APC, thereby stimulating the T cell.
22. The method according to 21, comprising introducing the stimulated T cell into the individual.
23. The method according to 21 or 22, wherein the T cell is autologous to the individual
24. A method of stimulating an antigen presenting cell (APC), the method comprising: contacting an APC with a Dectin-2 stimulating composition comprising one or more Dectin-2 stimulating agents selected from:

(i) a non-plant derived naturally existing ligand for Dectin-2;

(ii) a synthetic Dectin-2 stimulating glycopolymer; and

(iii) a Dectin-2 stimulating anti-Dectin-2 antibody,

at a dose and for a period of time sufficient to enhance Dectin-2 signaling in the APC, thereby generating a stimulated APC.
25. The method according to 24, comprising contacting the stimulated APC with a cancer antigen to produce an antigen-contacted APC.
26. The method according to 25, wherein the cancer antigen is present in a cancer cell lysate or is part of a cancer cell.
27. The method according to any of 24-26, comprising introducing the stimulated APC or the antigen-contacted APC into an individual.
28. The method according to 25 or 26, wherein the cancer antigen is from an individual with cancer and the method comprises introducing the antigen-contacted APC into the individual.
29. The method according to 27 or 28, wherein APC is autologous to the individual.
30. The method according to any of 25-29, comprising contacting a T cell with the antigen-contacted APC.
31. The method according to 30, comprising introducing the contacted T cell into an individual.
32. The method according to 31, wherein the T cell is autologous to the individual.
33. The method according to 31 or 32, wherein the antigen-contacted APC is autologous to the individual.
34. The method according to any of 27-33, wherein the individual has cancer.
35. A multivalent Dectin-2 stimulating agent, comprising:

(a) an agent that binds to Dectin-2 and stimulates Dectin-2 signaling; and

(b) an immunomodulatory agent or a targeting agent that specifically binds to a cancer antigen,

wherein (a) and (b) are conjugated to one another.

36. The multivalent Dectin-2 stimulating agent of 35, wherein (a) is an anti-Dectin-2 antibody or an antigen-binding region thereof.
37. The multivalent Dectin-2 stimulating agent of 35, wherein (a) is a glycopolymer that binds to Dectin-2.
38. The multivalent Dectin-2 stimulating agent of 35, wherein (a) is a natural Dectin-2 ligand.
39. The multivalent Dectin-2 stimulating agent of 38, wherein the natural Dectin-2 ligand comprises mannan polysaccharide, an oligomannose glycan, and/or a fungal cell wall extract.
40. The multivalent Dectin-2 stimulating agent of any one of 35-39, wherein (b) is an immunomodulatory agent.
41. The multivalent Dectin-2 stimulating agent of 40, wherein (b) is a cytokine selected from: IL-I, IL-2, IL-3, IL-4, IL-6, IL-7, IL-9, IL-10, IL-12, IL-15, IL-18, IL-21, IFN-α, IFN-β, IFN γ, G-CSF, TNFα, and GM-CSF.
42. The multivalent Dectin-2 stimulating agent of 40, wherein (b) is a growth factor.
43. The multivalent Dectin-2 stimulating agent of 40, wherein (b) is a stimulatory ligand for a pattern recognition receptor (PRR).
44. The multivalent Dectin-2 stimulating agent of 40, wherein (b) is a checkpoint blockade agent.
45. The multivalent Dectin-2 stimulating agent of any one of 35-39, wherein (b) is a targeting agent that specifically binds to a cancer antigen.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., room temperature (RT); base pairs (bp); kilobases (kb); picoliters (ph; seconds (s or sec); minutes (m or min); hours (h or hr); days (d); weeks (wk or wks); nanoliters (nl); microliters (ul); milliliters (ml); liters (L); nanograms (ng); micrograms (ug); milligrams (mg); grams ((g), in the context of mass); kilograms (kg); equivalents of the force of gravity ((g), in the context of centrifugation); nanomolar (nM); micromolar (uM), millimolar (mM); molar (M); amino acids (aa); kilobases (kb); base pairs (bp); nucleotides (nt); intramuscular (i.m.); intraperitoneal (i.p.); subcutaneous (s.c.); and the like.

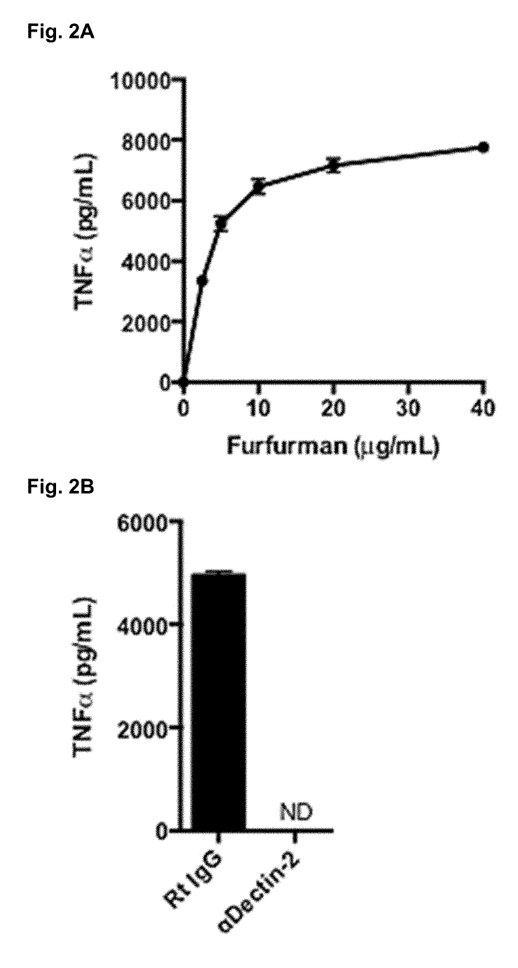

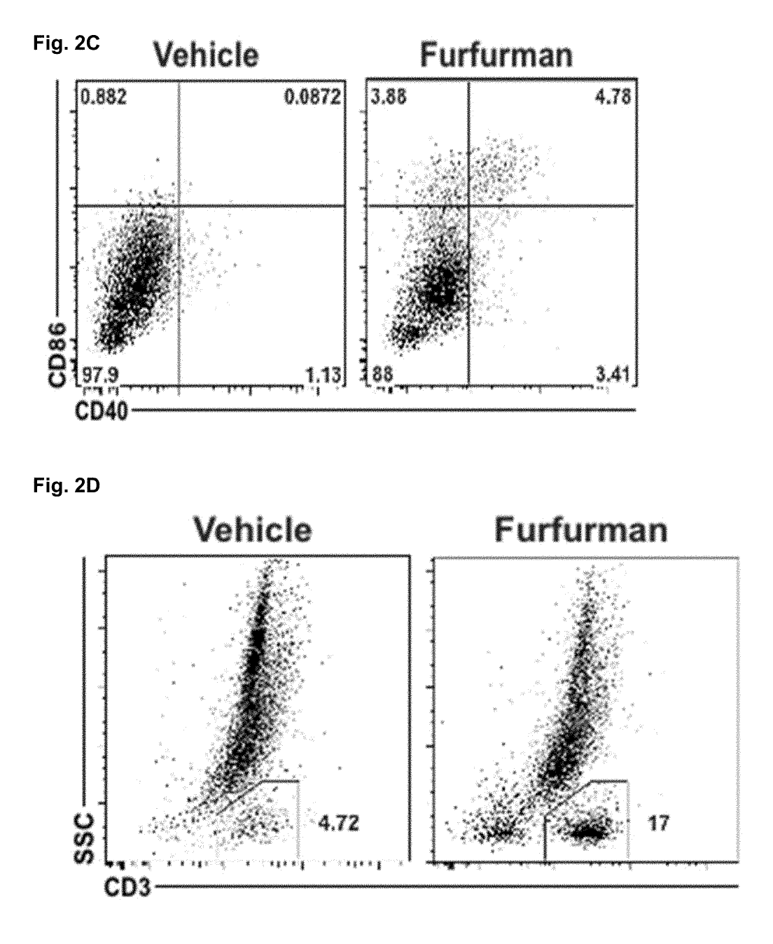

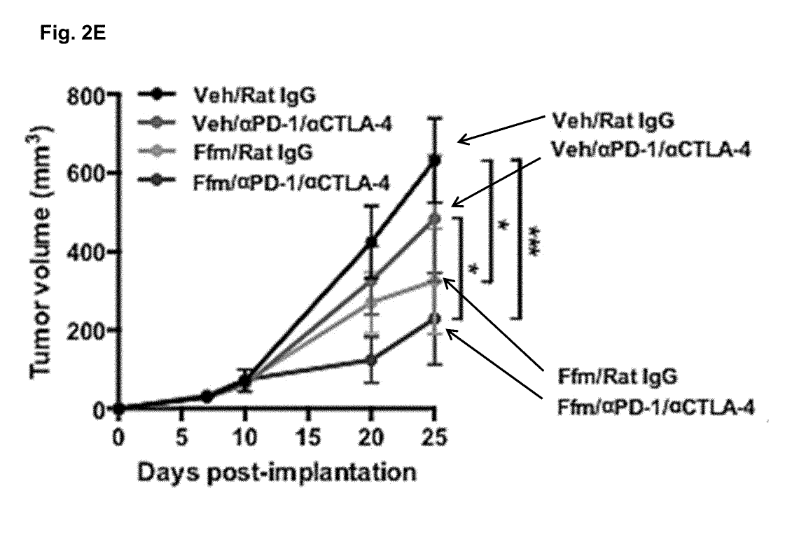

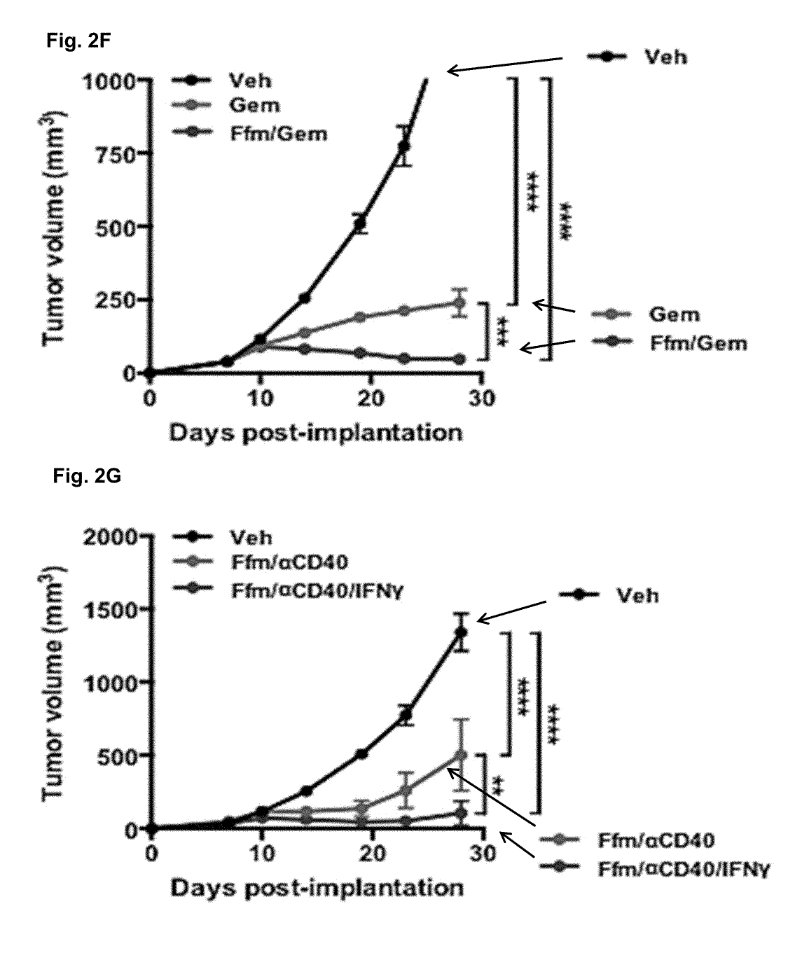

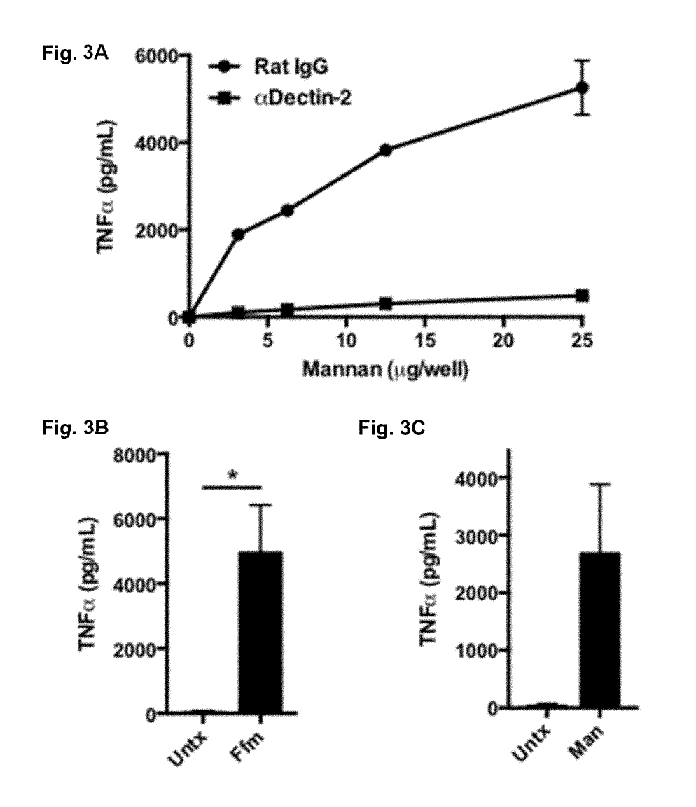

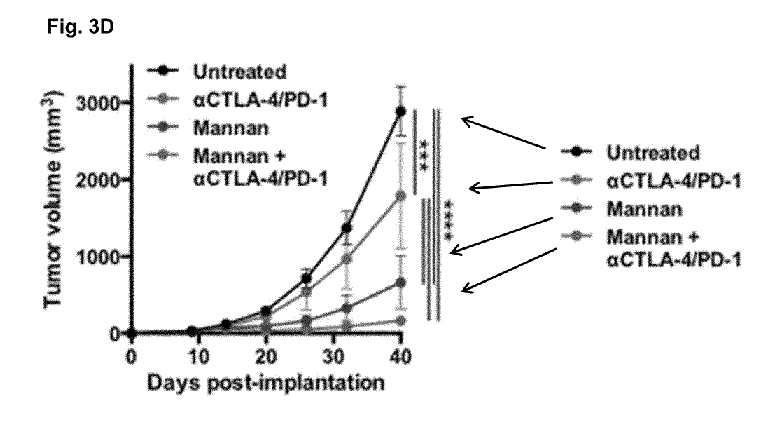

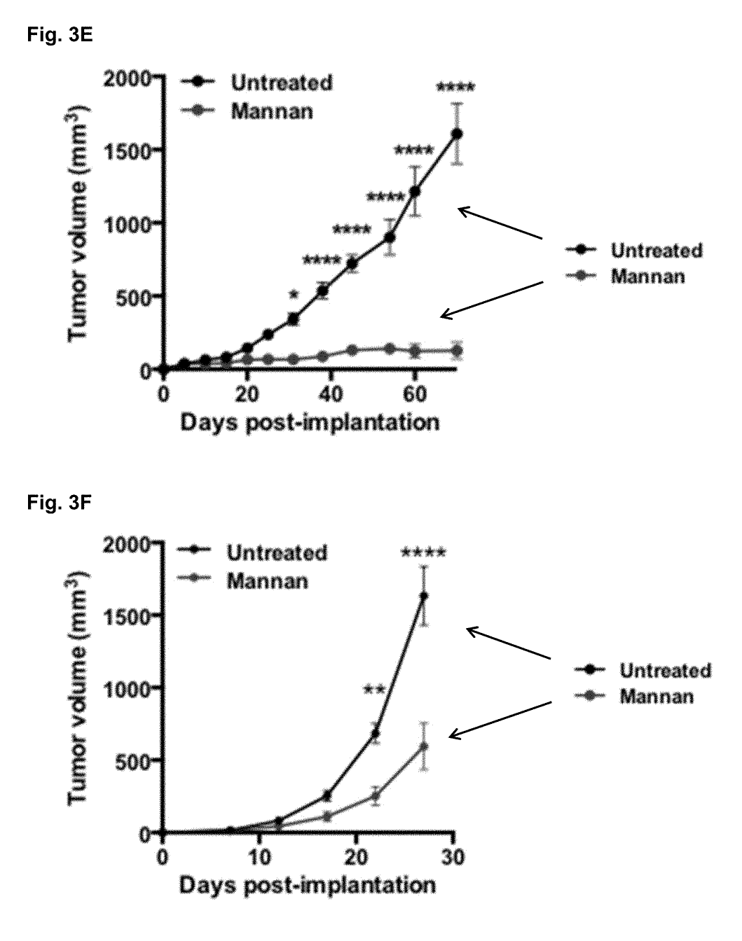

The experiments below demonstrate the development of multiple strategies to activate myeloid cells (e.g., tumor-associated myeloid (TAM) cells such as macrophages and dendritic cells) through Dectin-2 engagement. The experiments below show that Dectin-2 stimuli reprogram immunosuppressive TAM cells into proinflammatory cells that induce antitumor immune responses and support (e.g., synergize with) chemotherapy (e.g., conventional chemotherapy) and other immunotherapies (e.g. checkpoint inhibitors, CD40 agonists), resulting in tumor regression (FIG. 2E-2G, FIG. 3D-F). These findings have major implications for immuno-oncology (e.g., to treat cancers like PDAC that remain refractory to most therapeutic interventions tested to date).

### Example 1: Dectin-2 Expression

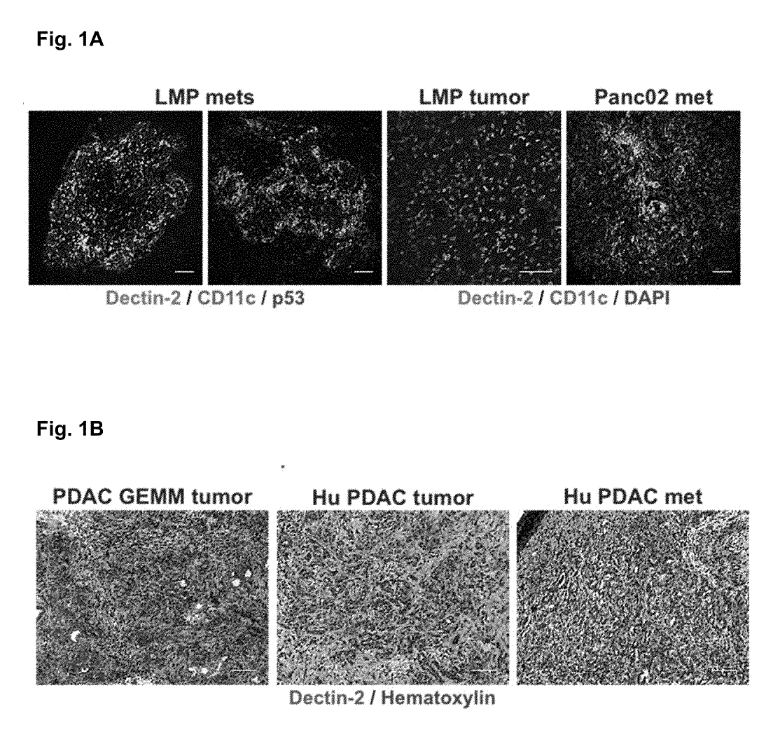

Tumor-associated myeloid (TAM) cells (tumor associated macrophages and dendritic cells (DC)) expressed high levels of Dectin-2 (FIG. 1A, FIG. 1B), a pattern recognition receptor (PRR) required for the induction of effective adaptive immune responses in various infectious diseases. This C-type lectin receptor, a class of carbohydrate binding proteins, has been shown to recognize a diverse range of components containing multiple terminal mannose residues from fungi and other pathogens. Consistent with this, Dectin-2 selectively binds high-mannose glycans in a carbohydrate array (e.g., see McGreal et al., Glycobiology. 2006 May; 16(5):422-30).

### Example 2: Treatment with Natural Dectin-2 Agonists

Various pathogens, including several fungal species like the opportunistic pathogen Malassezia furfur harbor Dectin-2-activating factors. In the experiments presented here (FIG. 2A-2G), a commercially available cell wall extract of M. furfur (furfurman; Invivogen) activated tumor-associated myeloid (TAM) cells in a Dectin-2-dependent fashion, which led to proinflammatory cytokine production and costimulatory molecule expression by the TAM cells (FIG. 2A-2C). Repeated i.v. injection of the Dectin-2 agonist from M. furfur was well tolerated in mice.

Consistent with these data, the studies in murine PDAC models indicated that intratumoral injection of a natural Dectin-2 agonist induces T cell infiltration (FIG. 2D) and inhibits tumor growth (FIG. 2E). Furthermore, when combined with conventional chemotherapy (i.e. gemcitabine) or more established cancer immunotherapies (i.e. checkpoint inhibitors, CD40 agonists), Dectin-2 stimulation led to tumor regression and even tumor clearance in some cases (FIG. 2E-2G). Dectin-2 agonists can be combined with other adjuvants to further enhance TAM activation. For example, we find that cells stimulated with both a Dectin-2 agonist and the cytokine IFNγ express very high levels of proinflammatory cytokines, cytotoxic mediators, and costimulatory molecules, including CD40. Correspondingly, combining these adjuvants with CD40 agonistic antibody treatment consistently led to tumor regression in vivo (FIG. 2G).

FIG. 3A-3F demonstrate that natural Dectin-2 ligands such as S. cerevisiae mannan (e.g., extract available from Sigma Aldrich) activate tumor-associated myeloid cells (e.g., human cells) and induce therapeutic antitumor immune responses. Mannan was active when delivered systemically and treated multiple tumor types (e.g., pancreatic, lung, and colon cancer). (FIG. 3A) TNFα production by PDAC TAM that were pretreated with the indicated antibodies and then stimulated overnight with plate-bound S. cerevisiae mannan. (FIG. 3B, FIG. 3C) TNFα production by human monocytes that were pretreated with GM-CSF and then stimulated with furfurman (FIG. 3B) or mannan (FIG. 3C). Mean±SEM for n=3 donors shown. (FIG. 3D-3F) Mice bearing s.c. PDAC (FIG. 3D), lung adenocarcinoma (FIG. 3E), or CT26 colon carcinoma were treated with mannan (i.v.) and/or a combination of αCTLA-4 and αPD-1 antibodies (i.p.) starting 6-9 days after tumor implantation. Mean tumor volumes±SEM are shown (n=3-5 per group). *, p<0.05; **, p<0.01; ***, p<0.001; ****, p<0.0001 by unpaired Student's t-test (B) or two-way ANOVA with post hoc Tukey's test (FIG. 3D-3F).

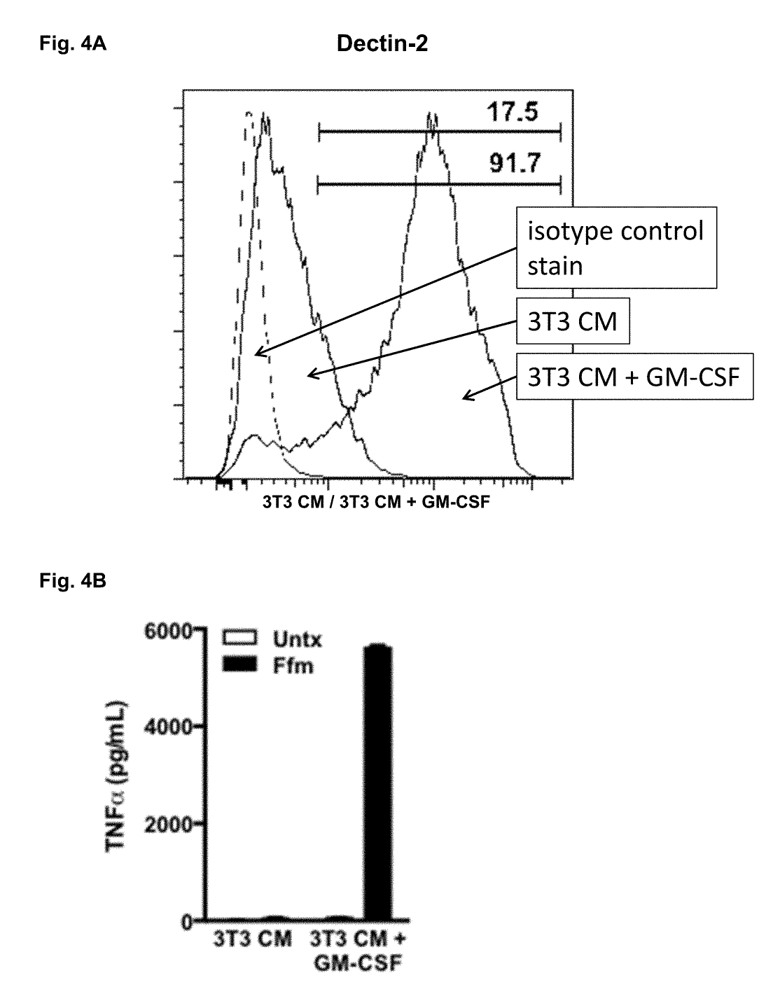

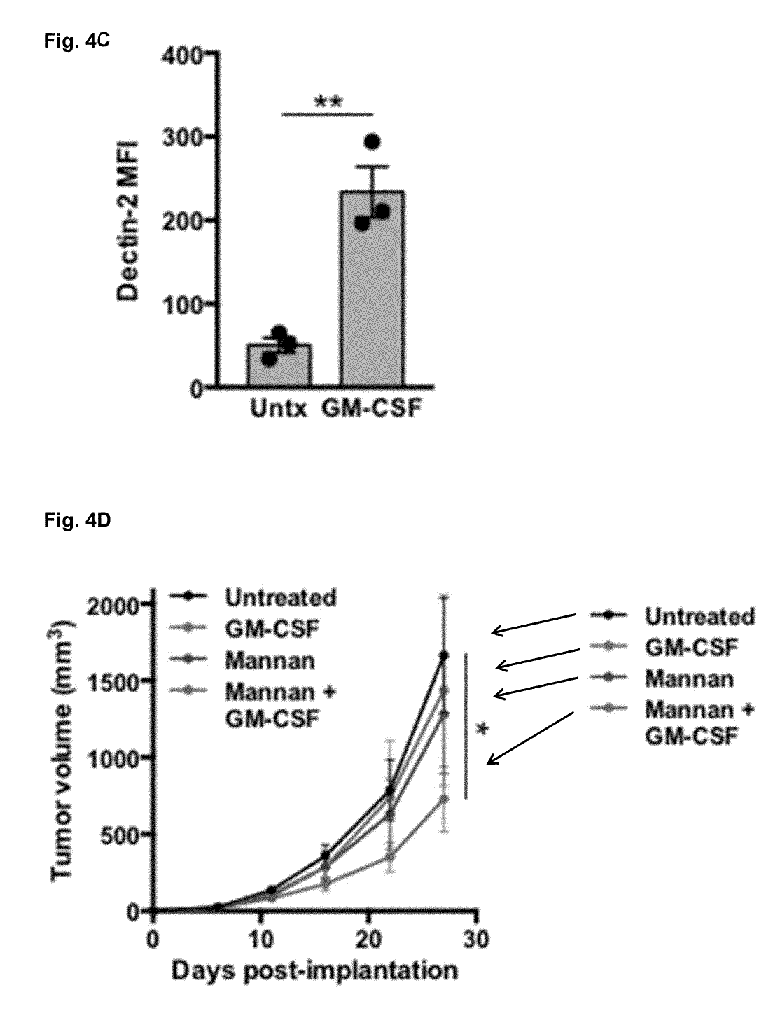

FIG. 4A-4D demonstrate that Dectin-2 expression can be induced with GM-CSF to make cells/tumors more responsive to Dectin-2 stimuli in both mouse and human systems. In other words, GM-CSF induced Dectin-2 expression and sensitized tumors to Dectin-2 stimuli. (FIG. 4A-4C) Murine (FIG. 4A, FIG. 4B) and human (FIG. 4C) monocytes were cultured for 24 hr in media supplemented or not with GM-CSF (50 ng/mL) prior to flow cytometric analysis of Dectin-2 expression (FIG. 4A, FIG. 4C) or stimulation with furfurman and analysis of GM-CSF production. (FIG. 4C) Mean MFI±SEM for n=3 donors displayed. (FIG. 4D) Mice bearing s.c. CT26 tumors were treated with mannan (i.v.) and/or GM-CSF (i.t.) starting on day 6 post-tumor implantation. Mean tumor volumes±SEM are displayed (n=3-4 per group). *, p<0.05; **, p<0.01 by Student's t-test (FIG. 4B) or two-way ANOVA with post hoc Tukey's test (FIG. 4C).

### Example 3: Treatment with Class I Alpha-Mannosidase Inhibitors

Dectin-2 recognizes various pathogen components containing multiple terminal mannose residues and reacts strongly with high-mannose type glycans. High-mannose glycans are common intermediate glycan species generated during N-linked glycosylation of proteins in eukaryotic cells. In mammalian cells, these high-mannose glycans are further processed into complex or hybrid type N-glycans—a process which requires the action of various mannosidases that cleave terminal mannose residues from the initial high-mannose precursor, Man₉GlcNAc₂ (Man-9).

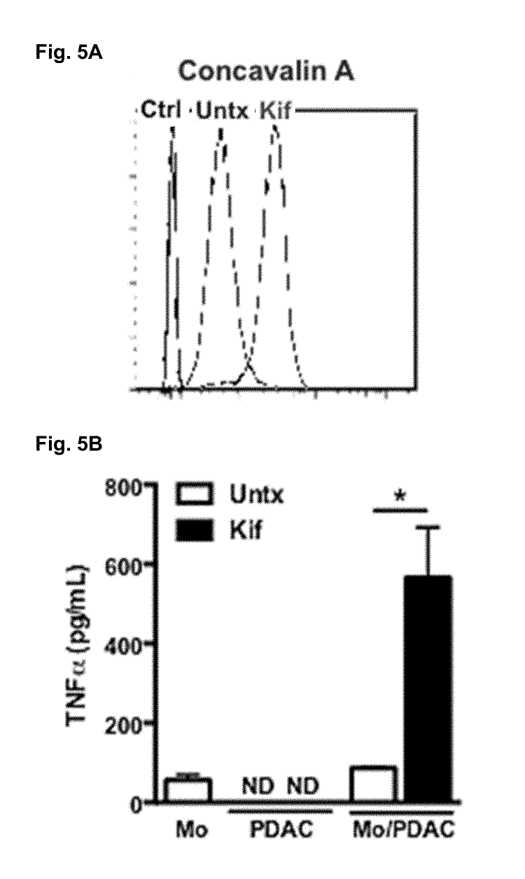

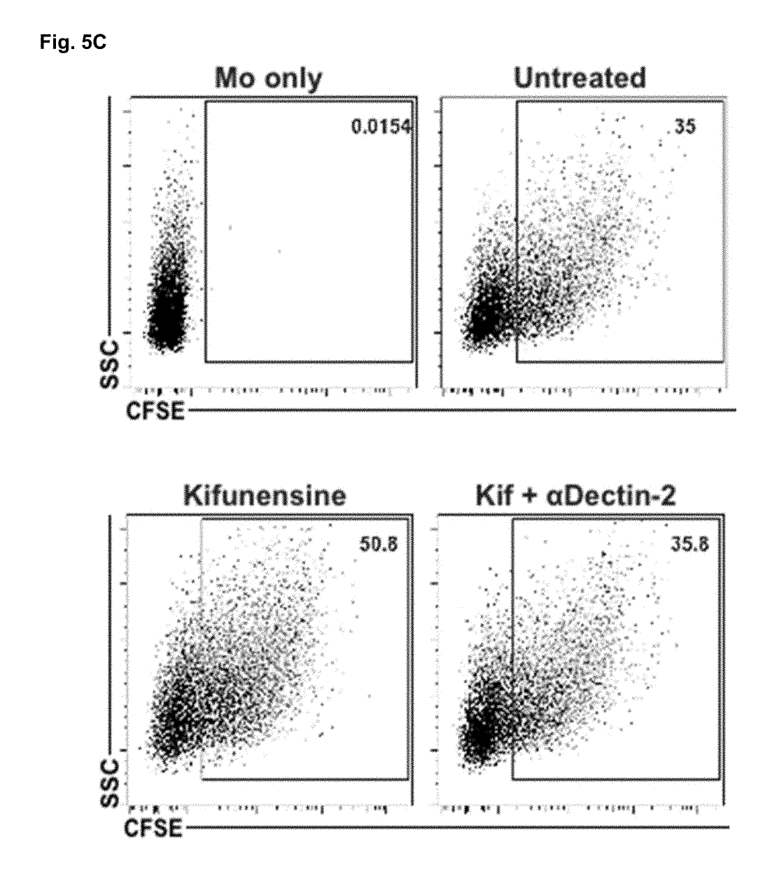

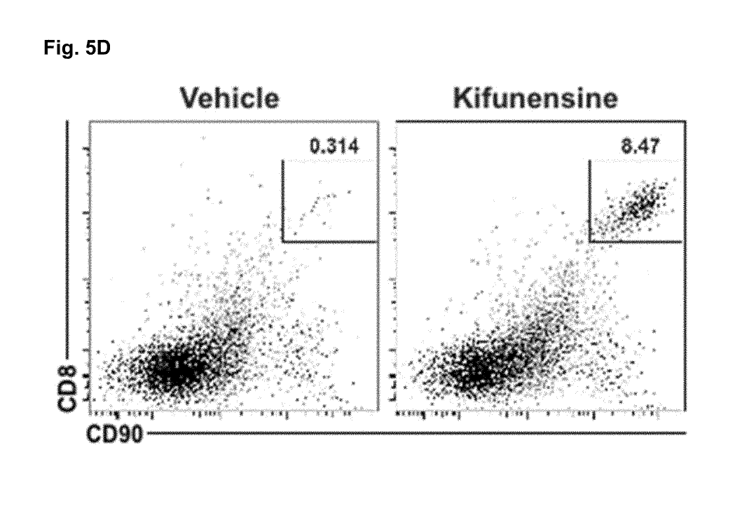

Treating tumor cells with kifunensine (an example of a small molecule alpha-mannosidase class 1 (α-mannosidase I) inhibitor) led to a sharp increase in high-mannose glycans on the cell surface (FIG. 5A). The tumor cells subsequently activated tumor-associated myeloid cells (TAM cells) (e.g., tumor associated dendritic cells and macrophages) in a Dectin-2-dependent fashion, inducing proinflammatory cytokine production and tumor cell uptake (FIG. 5B, FIG. 5C). In vivo, kifunensine treatment similarly increased high-mannose glycan display by tumor cells and led to T cell infiltration into tumors (FIG. 5D). These data support the usage of mannosidase inhibitors to augment tumor immunogenicity.

### Example 4: Treatment with Dectin-2-Activating Antibodies and Glycoconjugates

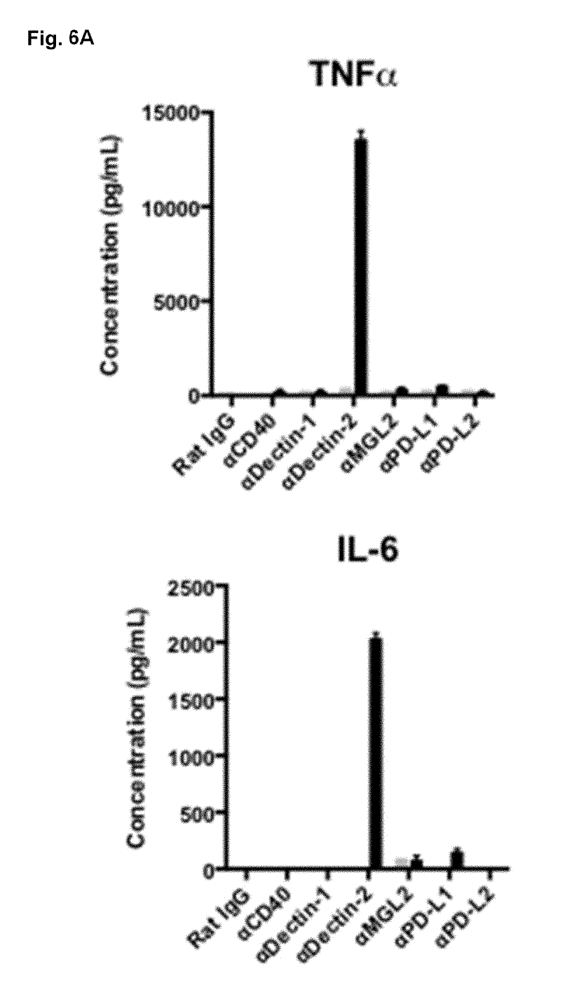

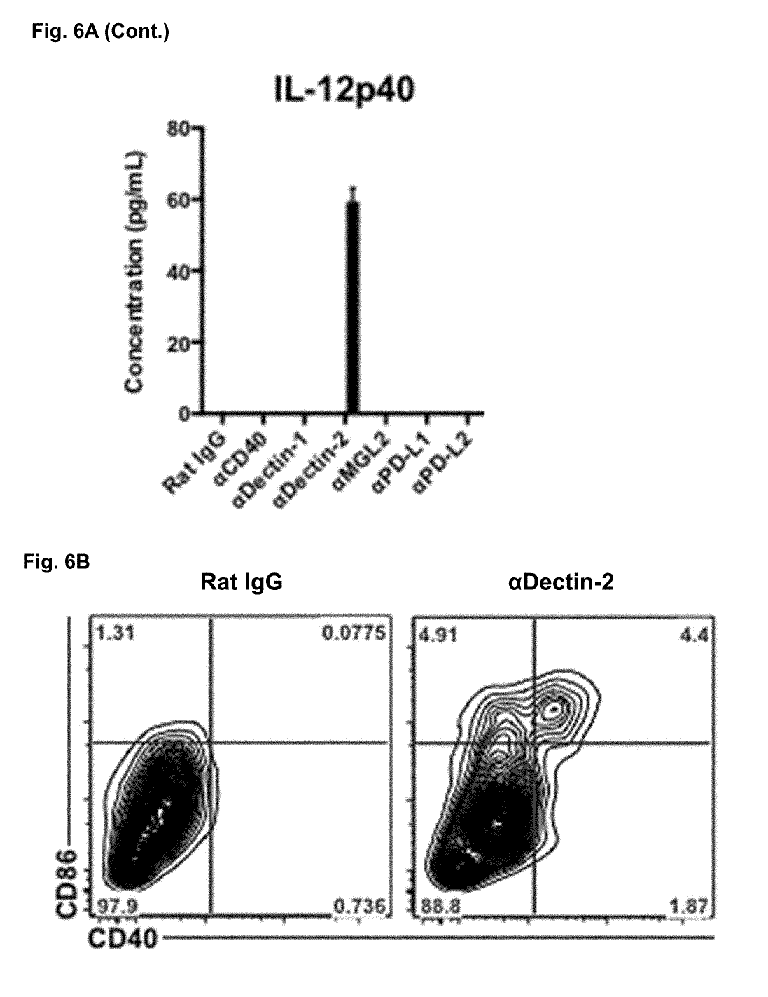

The data presented here show that tumor associated macrophages (TAM) were strongly activated by a particulate cell wall extract from M. furfur as well as immobilized (i.e. plate-bound) anti-Dectin-2 antibodies (FIG. 6A, FIG. 6B). These results are consistent with Dectin-2 requiring receptor clustering for signal transduction, and indicate that TAM may be activated by antibodies and glycoconjugates that induce sufficient Dectin-2 clustering (which can be achieved in a number of ways, including the use of direct Dectin-2 stimulating agent such as multivalent Dectin-2 stimulating agents (described above, e.g., such as high-mannose-modified antibody glycoconjugates) and/or the use of naturally existing or synthetic glycopolymers such as glycopolypeptides, e.g., an oligomannose glycopolypeptide (e.g., a mannobiose-rich glycoprotein, e.g., an O-linked and/or N-linked mannobiose-rich glycoprotein).

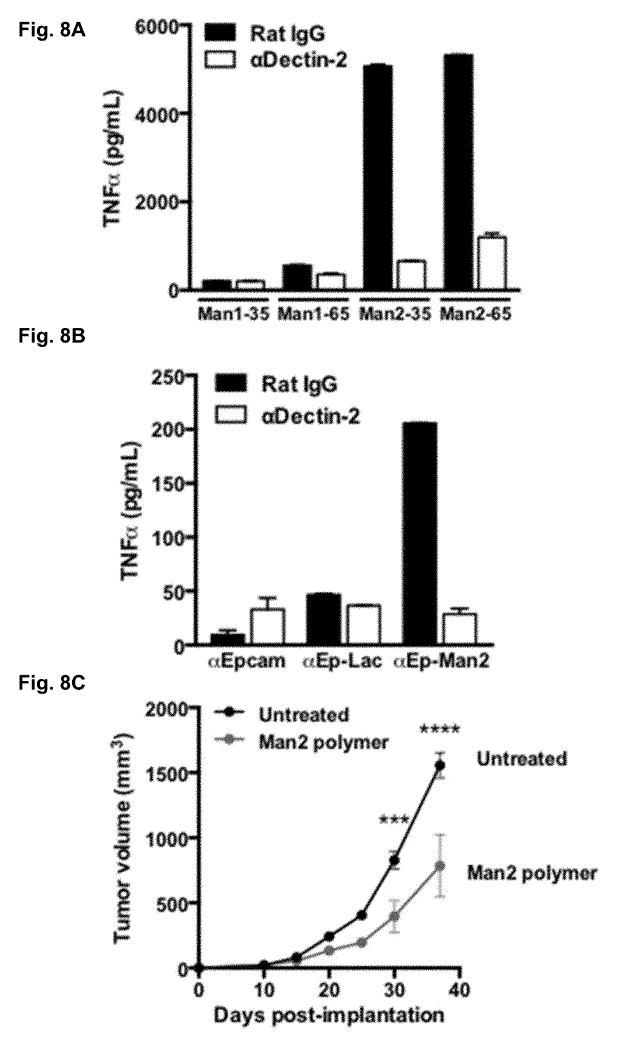

FIG. 8A-8C demonstrate that mannobiose glycopolymers and antibody-glycopolymer conjugates activated cells through Dectin-2, and that mannobiose glycopolymers are therapeutically active. Synthetic mannobiose glycopolymers and glycoconjugates activated myeloid cells for therapeutic effect. (FIG. 8A, FIG. 8B) TNFα production by PDAC TAM that were pretreated with the indicated antibodies and then stimulated with (FIG. 8A) mannose (Man1) or mannobiose (Man2) glycopolymers of different glycan densities (35% or 65%) or (FIG. 8B) αEpCAM antibodies coupled to lactose (Lac) or Man2 glycopolymers. (FIG. 8C) Mice bearing s.c. PDAC tumors were treated or not with Man2 glycopolymers (i.v.) starting 10 d following tumor implantation. Mean tumor volumes±SEM are shown (n=3-5 per group). ***, p<0.001; ****, p<0.0001 by two-way ANOVA with post hoc Tukey's test.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1-45. (canceled)

46. A conjugate comprising:
(a) a synthetic mannobiose glycopolypeptide, or an anti-Dectin-2 antibody or antigen fragment thereof, that binds to Dectin-2 and stimulates Dectin-2 signaling, and
(b) an immunomodulatory agent, a targeting agent that specifically binds to a cancer antigen, or a toll-like receptor (TLR) agonist,
wherein (a) and (b) are conjugated to each other.

47. The conjugate of claim 46, wherein (b) is a TLR agonist.

48. The conjugate of claim 47, wherein the TLR agonist is a TLR 7/8 agonist.

49. The conjugate of claim 46, wherein (a) is a synthetic mannobiose glycopolypeptide.

50. The conjugate of claim 49, wherein the synthetic mannobiose glycopeptide comprises from about 8 to about 300 amino acids.

51. The conjugate of claim 49, wherein the synthetic mannobiose glycopolypeptide has at least 35% glycan density.

52. The conjugate of claim 49, wherein the synthetic mannobiose glycopolypeptide comprises a mucin-like glycoprotein with mannobiose.

53. The conjugate of claim 46, wherein (b) is a targeting agent that specifically binds to a cancer antigen selected from the group consisting of CD19, CD20, CD22, CD24, CD25, CD30, CD33, CD38, CD44, CD40, CD47, CD52, CD56, CD70, CD96, CD97, CD99, CD123, CD279 (PD-1), CD274 (PD-L1), EpCAM, EGFR, HER2, CD117, C-Met, PTHR2, and HAVCR2 (TIM3).

54. The conjugate of claim 46, wherein (b) is a targeting agent that specifically binds to a cancer antigen, and the targeting agent that specifically binds to a cancer antigen is a Fab, scFv, or scDb.

55. The conjugate of claim 46, wherein (b) is a targeting agent that specifically binds to a cancer antigen, and the targeting agent that specifically binds to a cancer antigen is an antibody.

56. The conjugate of claim 55, wherein the antibody is selected from the group consisting of cetuximab, panitumumab, rituximab, trastuzumab, pertuzumab, alemtuzumab, brentuximab, tositumomab, ibritumomab, gemtuzumab, ibritumomab, and edrecolomab.

57. The conjugate of claim 55, wherein the antibody is an antibody that binds HER2.

58. The conjugate of claim 55, wherein the antibody is an antibody that binds EpCAM.

59. The conjugate of claim 46, wherein (b) is an immunomodulatory agent.

60. The conjugate of claim 59, wherein the immunomodulatory agent is an anti-CTLA4 antibody or antigen-binding region thereof, an anti-PD-1 agent or antigen-binding region thereof, an anti-PD-L1 agent or antigen-binding region thereof, an anti-CD40 antibody or antigen-binding region thereof, an anti-CD47 antibody or antigen-binding region thereof, or an anti-SIRPA antibody or antigen-binding region thereof.

61. The conjugate of claim 46, wherein (a) is a synthetic mannobiose glycopolypeptide, (b) is TLR agonist, and (a) or (b) is also conjugated to an antibody.

62. A method for stimulating Dectin-2 comprising:
providing a conjugate to a myeloid cell, the conjugate comprising (a) a synthetic mannobiose glycopolypeptide, or an anti-Dectin-2 antibody or antigen fragment thereof, that binds to Dectin-2 and stimulates Dectin-2 signaling, and (b) an immunomodulatory agent, a targeting agent that specifically binds to a cancer antigen, or a toll-like receptor (TLR) agonist, wherein (a) and (b) are conjugated to each other.

63. A method for treating cancer comprising
administering to a patient in need of cancer treatment an effective amount of a conjugate, the conjugate comprising (a) a synthetic mannobiose glycopolypeptide, or an anti-Dectin-2 antibody or antigen fragment thereof, that binds to Dectin-2 and stimulates Dectin-2 signaling, and (b) an immunomodulatory agent, a targeting agent that specifically binds to a cancer antigen, or a toll-like receptor (TLR) agonist, wherein (a) and (b) are conjugated to each other.

64. The method of claim 63, wherein the cancer is selected from the group consisting of pancreatic cancer, lung cancer, oral cancer, colon cancer, and a KRAS-mutant cancer.

65. The method of claim 63, wherein the conjugate is co-administered with a cancer therapeutic drug.


SEQUENCE LISTING (1 sequence)

SEQ ID NO: 1 - protein, 209 aa, Homo sapiens
MMQEQQPQSTEKRGWLSLRLWSVAGISIALLSACFIVSCVVTYHFTYGETGKRLSELHSYHSSLTCFSEGTKVPAWGCCPASWKSFGSSCYFISSEEKVWSKSEQNCVEMGAHLVVFNTEAEQNFIVQQLNESFSYFLGLSDPQGNNNWQWIDKTPYEKNVRFWHLGEPNHSAEQCASIVFWKPTGWGWNDVICETRRNSICEMNKIYL